(19) United States
(12) Patent Application Publication
(10) Pub. No.: US 2012/0121537 A1
(43) Pub. Date: May 17, 2012

(54) Methods and Compositions for Inhibiting Hepatitis C Virus Replication

(76) Inventors: Chaomin Sun, Albany, CA (US); James Harrison Doudna Cate, Berkeley, CA (US)

(21) Appl. No.: 13/144,067

(22) PCT Filed: Jan. 11, 2010
(86) PCT No.: PCT/US2010/020650

§ 371 Date: Oct. 26, 2011

Related U.S. Application Data
(60) Provisional application No. 61/144,032, filed on Jan. 12, 2009.

(51) Int. Cl. A61K 38/20; C12N 15/63; C12N 5/10; C12N 1/19; A61K 31/7088; C07K 19/00; C12N 9/96; A61K 38/02; C12N 5/071; A61K 38/21; G01N 33/573; C07K 4/00; C07K 14/18; C08H 1/00; A61P 31/14; C12N 15/62

(52) U.S. Cl. 424/85.2; 536/23.4; 536/23.2; 435/320.1; 435/325; 435/419; 435/348; 435/254.2; 435/366; 435/363; 435/353; 435/354; 435/367; 435/369; 435/358; 435/365; 435/352; 435/356; 514/44 R; 530/350; 435/188; 530/387.3; 530/362; 530/395; 514/4.3; 435/375; 424/85.4; 435/7.8; 436/501; 530/300; 530/322; 525/54.1

(57) ABSTRACT

Compositions and methods for reducing hepatitis C virus (HCV) replication are provided. Also provided are compositions and methods of treating an HCV infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection.

### CROSS-REFERENCE

This application claims the benefit of U.S. Provisional Patent Application No. 61/144,032, filed Jan. 12, 2009, which application is incorporated herein by reference in its entirety.

### STATEMENT REGARDING FEDERALLY SPONSORED RESEARCH

This invention was made with government support under Grant No. P01-GM07373202 awarded by the National Institutes of Health. The government has certain rights in the invention.

### BACKGROUND

Hepatitis C virus (HCV) is a blood-borne pathogen that is estimated to infect 150-200 million people worldwide. Infection by HCV may be non-symptomatic, and can be cleared by patients, sometimes without medical intervention. However, the majority of patients develop a chronic HCV infection, which may lead to liver inflammation, scarring, and even to liver failure or liver cancer. In the United States alone, over 3 million people have a chronic infection.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., such patients are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is a positive-strand RNA virus whose genome is directly translated into a polyprotein that encodes several functional fragments required for the viral life cycle. The HCV polyprotein is cleaved into proteins important for replicating the RNA virus, and proteins necessary for viral packaging. HCV genomic RNA includes a 5′-nontranslated region that includes an Internal Ribosome Entry Site (IRES) that contributes to translation of the polyprotein. Hepatitis C virus encodes an IRES (HCV IRES) upstream of its polyprotein open reading frame that requires only translation initiation factors eIF2 and eIF3 to function in vitro. Biochemical and structural results indicate that the HCV IRES likely makes multiple contacts with both the 40S ribosomal subunit and eIF3 in translation initiation complexes.

### Literature

Ariumi et al. (2007) J. Virol. 81:13922; Boni et al. (2005) J. Biol. Chem. 280:17737; Lee et al. (2008) Nucl. Acids Res. 36:4708; Mamiya and Worman (1999) J. Biol. Chem. 274:15751; Owsianka and Patel (1999) 257:330; You et al. (1999) J. Virol. 73:2841.

### SUMMARY OF THE INVENTION

Compositions and methods for reducing hepatitis C virus (HCV) replication are provided. Also provided are compositions and methods of treating an HCV infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection.

### BRIEF DESCRIPTION OF THE DRAWINGS

FIG. 1A depicts primary structures of certain proteins; and FIG. 1B depicts in vitro interactions between various N-terminal fragments of the HCV core protein and the helicase domain of human DDX3X.

FIG. 2A schematically depicts the HCV replicon NNeo/C-5B. FIG. 2B depicts inhibition of HCV replicon replication in human Huh7 cells due to expression of an N-terminal fragment of the HCV core protein.

FIG. 3 depicts an amino acid sequence of a human DDX3X polypeptide (SEQ ID NO:1; GenBank NP_{—}001347; 662 amino acids; DEAD/H (Asp-Glu-Ala-Asp/His) box polypeptide 3; DDX3X).

FIG. 4 depicts amino acid sequences of HCV core proteins.

FIG. 5 depicts an amino acid sequence of a human IgG1 Fc polypeptide (SEQ ID NO:8). The Fc loop regions in boldface.

### DEFINITIONS

As used herein, the term “flavivirus” includes any member of the family Flaviviridae, including, but not limited to, Dengue virus, including Dengue virus 1, Dengue virus 2, Dengue virus 3, Dengue virus 4 (see, e.g., GenBank Accession Nos. M23027, M19197, A34774, and M14931); Yellow Fever Virus; West Nile Virus; Japanese Encephalitis Virus; St. Louis Encephalitis Virus; Bovine Viral Diarrhea Virus (BVDV); and Hepatitis C Virus (HCV); and any serotype, strain, genotype, subtype, quasispecies, or isolate of any of the foregoing. The term “hepatitis C virus,” or “HCV,” encompasses any of a number of genotypes, subtypes, or quasispecies, of HCV, including, e.g., genotype 1, including 1a and 1b, 2, 3, 4, 6, etc. and subtypes (e.g., 2a, 2b, 3a, 4a, 4c, etc.), and quasispecies.

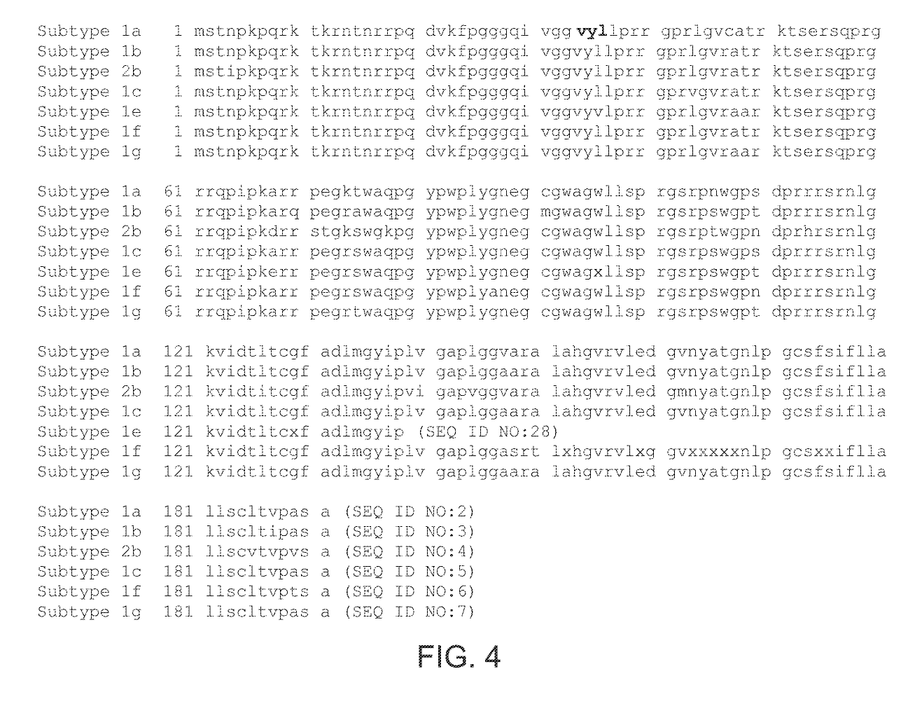

“HCV core protein” refers to the nucleocapsid protein of any serotype, strain, genotype, subtype, quasispecies, or isolate of HCV. For example, an HCV core protein can be from about 180 amino acids to about 190 amino acids in length, and can have an amino acid sequence having at least about 75%, at least about 80%, at least about 85%, at least about 90%, at least about 95%, at least about 98%, at least about 99%, or 100%, amino acid sequence identity to an amino acid sequence depicted in FIG. 4 (SEQ ID NOs:2-7).

The term “functional in vivo half-life” is used in its normal meaning, i.e. the time at which 50% of the biological activity of the polypeptide is still present in the body/target organ, or the time at which the activity of the polypeptide is 50% of the initial value. The functional in vivo half-life may be determined in an experimental animal, such as rat, mice, rabbit, dog or monkey. Furthermore, the functional in vivo half-life may be determined for a sample that has been administered intravenously or subcutaneously.

As an alternative to determining functional in vivo half-life, “serum half-life” may be determined, i.e. the time at which 50% of the polypeptide circulates in the plasma or bloodstream prior to being cleared. Determination of serum half-life is often more simple than determining the functional in vivo half-life and the magnitude of serum half-life is usually a good indication of the magnitude of functional in vivo half-life. Alternatively terms to serum half-life include “plasma half-life”, “circulating half-life”, “serum clearance”, “plasma clearance” and “clearance half-life”. The serum half-life may be determined as described above in connection with determination of functional in vivo half-life.

As used herein, the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells. An isolated polypeptide will in some embodiments be synthetic. “Synthetic polypeptides” are assembled from amino acids, and are chemically synthesized in vitro, e.g., cell-free chemical synthesis, using procedures known to those skilled in the art. In some embodiments, an isolated polypeptide will be purified. By “purified” is meant a compound of interest (e.g., a polypeptide) has been separated from components that accompany it in nature. “Purified” can also be used to refer to a polypeptide separated from components that can accompany it during production of the polypeptide (e.g., during synthesis in vitro, etc.). In some embodiments, a polypeptide is substantially pure when it is at least 75% by weight, free from organic molecules with which it is naturally associated or with which it is associated during production. In some embodiments, the polypeptide is at least 75%, at least 90%, at least 95%, or at least 99%, by weight, pure.

As used herein, the term “hepatic fibrosis,” used interchangeably herein with “liver fibrosis,” refers to the growth of scar tissue in the liver that can occur in the context of a chronic hepatitis infection.

The terms “individual,” “host,” “subject,” and “patient” are used interchangeably herein, and refer to a mammal, including, but not limited to, non-human primates (e.g., simians), and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”).

“Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

A “dominant negative” gene product, either RNA or protein, is one that interferes with the function of another gene product, either RNA or protein. The other gene product affected can be the same or different from the dominant negative gene product. Dominant negative gene products can be of many forms, including truncations, full length RNAs or proteins with point mutations or fragments thereof, or fusions of full-length wild type or mutant RNAs or proteins with other RNAs or proteins, respectively, or fragments of RNAs or proteins fused with other RNAs or proteins, respectively. The level of inhibition observed can range from low levels to complete inhibition.

The terms “linking” and “fusing” as used herein, refer to entities that are directly linked, or linked via an amino acid linker, the size and composition of which can vary, or linked via a chemical linker.

The terms “modulating replication” and “inhibiting replication” of the hepatitis C viral genome refers to the ability of a dominant negative gene to inhibit replication of the HCV genome Inhibition can occur during replication of the genomic or anti-genomic RNAs, or during translation of the genomic RNA into the HCV polyprotein.

The term “heterologous” is a relative term, which when used with reference to portions of a nucleic acid indicates that the nucleic acid comprises two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source or sources. The two or more nucleic acids are thus heterologous to each other in this context. When added to a cell, the recombinant nucleic acids would also be heterologous to the endogenous genes of the cell.

Similarly, a heterologous protein indicates that the protein comprises two or more subsequences that are not found in the same relationship to each other in nature (e.g., a “fusion protein,” where the two subsequences are encoded by a single nucleic acid sequence).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

An “expression vector” is a nucleic acid construct, generated recombinantly or synthetically, with a series of specified nucleic acid elements that permit transcription of a particular nucleic acid in a host cell, and optionally integration or replication of the expression vector in a host cell. The expression vector can be part of a plasmid, virus, or nucleic acid fragment, of viral or non-viral origin. Typically, the expression vector includes an “expression cassette,” which comprises a nucleic acid to be transcribed operably linked to a promoter. The term expression vector also encompasses naked DNA operably linked to a promoter.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms also apply to amino acid polymers in which one or more amino acid residues is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The term “substantially identical” indicates that two or more nucleotide sequences share a majority of their sequence. Generally, this will be at least about 90% of their sequence, or about 95% of their sequence. Another indication that sequences are substantially identical is if they hybridize to the same nucleotide sequence under stringent conditions (see, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997). Stringent conditions are sequence-dependent and will be different in different circumstances. Generally, stringent conditions are selected to be about 5° C. (or less) lower than the thermal melting point (Tm) for the specific sequence at a defined ionic strength and pH. The Tₘ of a DNA duplex is defined as the temperature at which 50% of the nucleotides are paired and corresponds to the midpoint of the spectroscopic hyperchromic absorbance shift during DNA melting. The Tₘ indicates the transition from double helical to random coil.

Typically, stringent conditions will be those in which the salt concentration is about 0.2×SSC at pH 7 and the temperature is at least about 60° C. For example, a nucleic acid of the invention or fragment thereof can be identified in standard filter hybridizations using the nucleic acids disclosed here under stringent conditions, which for purposes of this disclosure, include at least one wash in 0.2×SSC at a temperature of at least about 60° C., about 65° C., or about 70° C., for about 20 minutes, or equivalent conditions. For a polymerase chain reaction (PCR), an annealing temperature of about 5° C. below Tm is typical for low stringency amplification, although annealing temperatures may vary between about 32° C. and 72° C., e.g., 40° C., 42° C., 45° C., 52° C., 55° C., 57° C., or 62° C., depending on primer length and nucleotide composition or high stringency PCR amplification, a temperature at, or slightly (up to 5° C.) above, primer Tm is typical, although high stringency annealing temperatures can range from about 50° C. to about 72° C., depending on the primer and buffer conditions (Ahsen et al., Clin Chem. 47:1956-61, 2001). Exemplary cycle conditions for both high and low stringency amplifications include a denaturation phase of 90° C.-95° C. for from about 30 seconds to about 2 minutes, an annealing phase lasting from about 30 seconds to about 10 minutes, and an extension phase of about 72° C. for from about 1 minute to about 15 minutes.

The terms “identical” or percent “identity,” in the context of two or more nucleic acids, refer to two or more sequences or subsequences that are the same or have a specified percentage of nucleotides that are the same (i.e., at least 70% identity, preferably at least 75%, 80%, 85%, 90%, 95%, 96%, 97%, 98%, or 99% identity, over a specified region, when compared and aligned for maximum correspondence over a comparison window, or designated region as measured using a BLAST or BLAST 2.0 sequence comparison algorithms with default parameters described below, or by manual alignment and visual inspection. Such sequences are then said to be “substantially identical.” This definition also refers to the complement of a test sequence. In some embodiments, the identity exists over a region that is at least about 15, 20 or 25 nucleotides in length, or more preferably over a region that is 50-100 nucleotides in length.

For sequence comparison, one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 15 to 600, from about 20 to about 200, or from about 50 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the default parameters described herein, to determine percent sequence identity for the nucleic acids described herein. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always<0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide sequences would occur by chance. For example, a nucleic acid is considered similar to a reference sequence if the smallest sum probability in a comparison of the test nucleic acid to the reference nucleic acid is less than about 0.2, less than about 0.01, or less than about 0.001.

“Administering” an expression vector, nucleic acid, protein, or a delivery vehicle to a cell comprises transducing, transfecting, electroporating, translocating, fusing, phagocytosing, shooting or ballistic methods, etc., i.e., any means by which a protein or nucleic acid can be transported across a cell membrane and preferably into the nucleus of a cell.

A “delivery vehicle” refers to a compound, e.g., a liposome, toxin, or a membrane translocation polypeptide, which is used to deliver a polypeptide to a cell. Delivery vehicles can also be used to administer nucleic acids to a cell; delivery vehicles suitable for delivering a nucleic acid to a cell include, e.g., a lipid:nucleic acid complex, an expression vector, a virus, and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an inhibitor of HCV replication” includes a plurality of such inhibitors and reference to “the DDX3X polypeptide” includes reference to one or more DDX3X polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

Compositions and methods for reducing hepatitis C virus (HCV) replication are provided. Also provided are compositions and methods of treating an HCV infection; methods of reducing the incidence of complications associated with HCV and cirrhosis of the liver; and methods of reducing viral load, or reducing the time to viral clearance, or reducing morbidity or mortality in the clinical outcomes, in patients suffering from HCV infection.

It has been shown that certain fragments of the HCV core protein interact specifically with the cellular RNA helicase DDX3X in vitro, and that expression of an HCV core fragment blocks HCV replication in HCV-infected cells. Inhibition of the interaction between DDX3X and HCV core protein provides for inhibition of HCV replication in a cell Inhibition of HCV replication in a cell is useful for treating an HCV infection in an individual.

### Polypeptides

The present disclosure provides a DDX3X-binding HCV core fusion polypeptide. A subject DDX3X-binding HCV core fusion polypeptide comprises: a) a DDX3X-binding HCV core fragment; and b) a heterologous polypeptide. The present disclosure further provides a stabilized DDX3X-binding HCV core polypeptide fragment.

In some embodiments, a subject polypeptide (e.g., a subject DDX3X-binding HCV core fusion polypeptide; a subject stabilized DDX3X-binding HCV core polypeptide fragment) exhibits increased in vivo and/or serum half-life, compared to the in vivo or serum half-life of an HCV core polypeptide depicted in FIG. 4. For example, in some embodiments, a subject polypeptide (e.g., a subject DDX3X-binding HCV core fusion polypeptide; a subject stabilized DDX3X-binding HCV core polypeptide fragment) exhibits an in vivo and/or serum half-life that is at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 2.5-fold, at least about 5-fold, or at least about 10-fold, or more than 10-fold, greater than the in vivo or serum half-life of an HCV core polypeptide depicted in FIG. 4.

### DDX3X-Binding HCV Core Fusion Polypeptides

A subject DDX3X-binding HCV core fusion polypeptide, when present in a cell (e.g., an HCV-infected cell), inhibits HCV replication in the cell. For example, a subject DDX3X-binding HCV core fusion polypeptide inhibits HCV replication in a cell by at least about 10%, at least about 15%, at least about 20%, at least about 25%, at least about 30%, at least about 35%, at least about 40%, at least about 45%, at least about 50%, at least about 60%, at least about 70%, or at least about 80%, or more than 80%, compared to the level of HCV replication in the cell in the absence of the DDX3X-binding HCV core fusion polypeptide.

A subject DDX3X-binding HCV core fusion polypeptide can have a length of from about 25 amino acids to about 500 amino acids, e.g., from about 25 aa to about 50 aa, from about 50 aa to about 100 aa, from about 100 aa to about 150 aa, from about 150 aa to about 200 aa, from about 200 aa to about 250 aa, from about 250 aa to about 300 aa, from about 300 aa to about 400 aa, or from about 400 aa to about 500 aa.

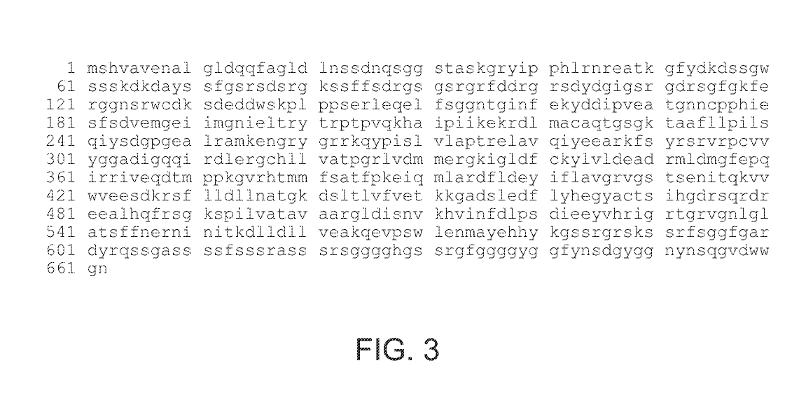

As used herein, “DDX3X” refers to a cellular RNA helicase that binds to HCV core protein and that has an amino acid sequence having at least about 75%, at least about 80%, at least about 85%, at least about 90%, at least about 95%, at least about 98%, or at least about 99%, amino acid sequence identity over a contiguous stretch of from about 300 amino acids (aa) to about 400 aa, from about 400 aa to about 450 aa, from about 450 aa to about 500 aa, from about 500 aa to about 550 aa, from about 550 aa to about 600 aa, or from about 600 aa to about 650 aa, up to 662 aa, of the amino acid sequence depicted in FIG. 3 (GenBank NP_{—}001347).

A subject DDX3X-binding HCV core fusion polypeptide comprises a DDX3X-binding HCV core polypeptide. In some embodiments, a DDX3X-binding fragment of HCV core protein includes at least amino acids 34-36 of HCV core protein, where the HCV core protein can be a core protein of HCV subtype 1a, HCV subtype 1b, subtype 2b, or any of subtypes 1c-1g. In some embodiments, a DDX3X-binding fragment of HCV core protein includes at least amino acids 34-36 of any one of the amino acid sequences depicted in FIG. 4, where amino acids 34-36 are Val-Tyr-Leu or Val-Tyr-Val. In the discussion below, the sequence “Val-Tyr-Leu” is noted; however, the sequence “Val-Tyr-Val” can also be used.

The amino acid sequences of HCV core proteins of various HCV subtypes are known in the art. See, e.g., GenBank ACE82312—HCV subtype 1a; GenBank BAG12384—HCV subtype 1b; GenBank AAP55704—HCV subtype 2b; GenBank BAA03581—HCV subtype 1c; GenBank AAC42169—HCV subtype 1e; GenBank AAC42170—HCV subtype 1f; and GenBank CAP45524—HCV subtype 1g.

A DDX3X-binding HCV core polypeptide can comprise amino acids 34-36 of HCV core protein, and from one to about 50 amino acids on the amino- and/or carboxyl-terminus of the Val-Tyr-Leu (or Val-Tyr-Val) peptide. Thus, for example a DDX3X-binding HCV core polypeptide can comprise, in addition to Val-Tyr-Leu, an amino acid sequence having at least about 75%, at least about 80%, at least about 85%, at least about 90%, at least about 95%, at least about 98%, or at least about 99%, or 100%, amino acid sequence identity to a stretch of from about 5 amino acids to about 33 amino acids of amino acids 1-33 of any one of the amino acid sequences depicted in FIG. 4.

As another example, a DDX3X-binding HCV core polypeptide can comprise, in addition to Val-Tyr-Leu, an amino acid sequence having at least about 75%, at least about 80%, at least about 85%, at least about 90%, at least about 95%, at least about 98%, or at least about 99%, or 100%, amino acid sequence identity to a stretch of from about 5 amino acids to about 10 amino acids, from about 10 amino acids to about 25 amino acids, from about 25 amino acids to about 50 amino acids, from about 50 to about 100 amino acids, or from about 100 amino acids to about 150 amino acids, of amino acids 37-190 of any one of the amino acid sequences depicted in FIG. 4.

As another example, a DDX3X-binding HCV core polypeptide can comprise, in addition to Val-Tyr-Leu, an amino acid sequence having at least about 75%, at least about 80%, at least about 85%, at least about 90%, at least about 95%, at least about 98%, or at least about 99%, or 100%, amino acid sequence identity to a stretch of from about 5 amino acids to about 33 amino acids of amino acids 1-33 of any one of the amino acid sequences depicted in FIG. 4; and an amino acid sequence having at least about 75%, at least about 80%, at least about 85%, at least about 90%, at least about 95%, at least about 98%, or at least about 99%, or 100%, amino acid sequence identity to a stretch of from about 5 amino acids to about 10 amino acids, from about 10 amino acids to about 25 amino acids, from about 25 amino acids to about 50 amino acids, from about 50 to about 100 amino acids, or from about 100 amino acids to about 150 amino acids, of amino acids 37-190 of any one of the amino acid sequences depicted in FIG. 4.

As another example, a DDX3X-binding HCV core polypeptide can have a length of from about 10 amino acids to about 100 amino acids, where the DDX3X-binding HCV core polypeptide comprises at least amino acids 34-36 of any one of the amino acid sequences depicted in FIG. 4, and where the DDX3X-binding HCV core polypeptide comprises an amino acid sequence having at least about 75%, at least about 80%, at least about 85%, at least about 90%, at least about 95%, at least about 98%, or at least about 99%, or 100%, amino acid sequence identity to a stretch of from about 10 amino acids to about 100 amino acids of any one of the amino acid sequences depicted in FIG. 4, and comprising amino acids 34-36 of any one of the amino acid sequences depicted in FIG. 4.

Non-limiting examples of DDX3X-binding HCV core polypeptides include, e.g.:

(SEQ ID NO: 9)

| | |
|---|---|
| 1) | MSTNPKPQRKTKRNTNRRPQDVKFPGGGQIVGGVYL; |

(SEQ ID NO: 10)

| | |
|---|---|
| 2) | VKFPGGGQIVGGVYLLPRRGPRLGVCAT; |

(SEQ ID NO: 11)

| | |
|---|---|
| 3) | STNPKPQRKTKRNTNRRPQDVKFPGGGQIVGGVYLLPRRGPRLG |
| | VCAT; |

(SEQ ID NO: 12)

| | |
|---|---|
| 4) | KTKRNTNRRPQDVKFPGGGQIVGGVYLLPRRGPRLGVCATRKT |
| | SER; |

(SEQ ID NO: 13)

| | |
|---|---|
| 5) | TNRRPQDVKFPGGGQIVGGVYLLPRRGPRLGVCATRKTSER; |

(SEQ ID NO: 14)

| | |
|---|---|
| 6) | KRNTNRRPQDVKFPGGGQIVGGVYLLPRRGPRLGVCAT; |

(SEQ ID NO: 15)

| | |
|---|---|
| 7) | RRPQDVKFPGGGQIVGGVYL; |

(SEQ ID NO: 16)

| | |
|---|---|
| 8) | MSTIPKPQRK TKRNTNRRPQ DVKFPGGGQI VGGVYL; |

(SEQ ID NO: 17)

| | |
|---|---|
| 9) | DVKFPGGGQI VGGVYLLPRR GPRLGVRATR; |

(SEQ ID NO: 18)

| | |
|---|---|
| 10) | DVKFPGGGQI VGGVYLLPRR GPRLGVRAAR; |
| and | |

(SEQ ID NO: 19)

| | |
|---|---|
| 11) | FPGGGQI VGGVYLLPRR GPRLGVRATR KTSER. |

As noted above, a subject DDX3X-binding HCV core fusion polypeptide comprises: a) a DDX3X-binding HCV core fragment; and b) a heterologous polypeptide. Suitable heterologous polypeptides include polypeptides that provide for increased solubility; polypeptides that provide for one or more of: increased in vivo stability; increased in vitro stability; increased in vivo solubility; increased in vivo resistance to proteolytic degradation; increased serum half-life; increased levels of in vivo protein expression; increased levels of in vitro protein expression; and the like.

Suitable heterologous polypeptides can have a length of from about 10 amino acids to about 250 amino acids, e.g., from about 10 amino acids to about 15 amino acids, from about 15 amino acids to about 20 amino acids, from about 20 amino acids to about 25 amino acids, from about 25 amino acids to about 50 amino acids, from about 50 amino acids to about 75 amino acids, from about 75 amino acids to about 100 amino acids, from about 100 amino acids to about 150 amino acids, from about 150 amino acids to about 200 amino acids, or from about 200 amino acids to about 250 amino acids.

Suitable heterologous polypeptides include, but are not limited to, glutathione-S-transferase; a chaperone polypeptide; an Fc domain polypeptide (see, e.g., U.S. Patent Publication No. 2009/0012272); a salvage receptor binding epitope of the Fc region of an IgG (see, e.g., U.S. Pat. No. 5,869,046); a peptibody; an Fc internal peptibody (e.g., as described in U.S. Pat. Nos. 7,442,778); variants of O-alkylguanine-DNA alkyltransferase (see, e.g., WO 2005/085431); a HaloTag (see, e.g., Los et al. (2008)ACS Chem. Biol. 3:373); a ubiquitin-like protein (e.g., Sumo; Nedd8; Rad23; etc.) an antibody or antibody fragment; a macroglobulin (e.g., α₂-macroglobulin); albumin; and variants of green fluorescent protein.

An exemplary salvage receptor binding epitope comprises the sequence:

PKNSSMISNTP (SEQ ID NO:20), and optionally further comprises a sequence selected from:

| | |
|---|---|
| HQSLGTQ, | (SEQ ID NO: 21) |
| HQNLSDGK, | (SEQ ID NO: 22) |
| HQNISDGK, | (SEQ ID NO: 23) |
| or | |
| VISSHLGQ. | (SEQ ID NO: 24) |

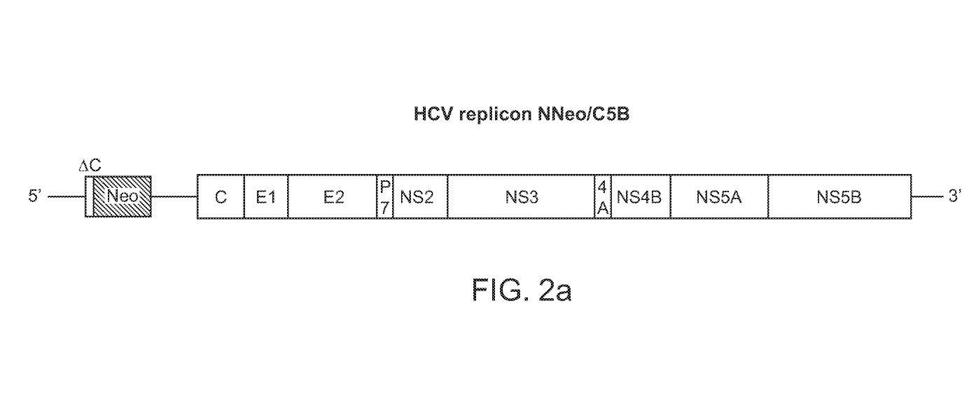

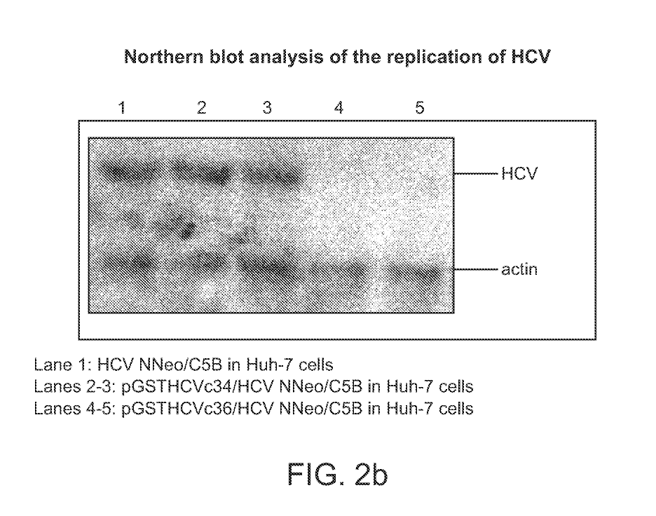

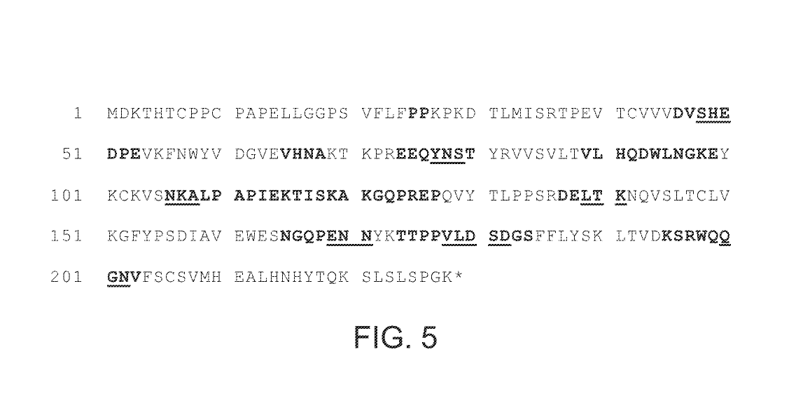

Both native Fc's and Fc variants are suitable Fc domains for use within the scope of this invention. A native Fc may be extensively modified to form an Fc variant in accordance with this invention, provided binding to the salvage receptor is maintained; see, for example WO 97/34631 and WO 96/32478. In such Fc variants, one may remove one or more sites of a native Fc that provide structural features or functional activity not required by the fusion molecules of this invention. One may remove these sites by, for example, substituting or deleting residues, inserting residues into the site, or truncating portions containing the site. The inserted or substituted residues may also be altered amino acids, such as peptidomimetics or D-amino acids. Fc variants can also be used. Exemplary Fc variants include molecules and sequences as described in U.S. Patent Publication No. 2009/0012272. Sites involved in disulfide bond formation are removed. Such removal may avoid reaction with other cysteine-containing proteins present in the host cell used to produce the molecules of the invention. For this purpose, the cysteine-containing segment at the N-terminus may be truncated or cysteine residues may be deleted or substituted with other amino acids (e.g., alanyl, seryl). In particular, one may truncate the N-terminal 20-amino acid segment of an Fc domain as described in U.S. Patent Publication No. 2009/0012272 or delete or substitute the cysteine residues at positions 7 and 10 of SEQ ID NO: 599 as described in U.S. Patent Publication No. 2009/0012272. SEQ ID NO:599 as described in U.S. Patent Publication No. 2009/0012272 is reproduced in FIG. 5 (SEQ ID NO:8). In FIG. 5, the Fc loop regions are in boldface. Any, or all of the sites shown in boldface are suitable for full or partial replacement by or insertion of peptide sequences; internal sites of particular interest are underlined. Potential loop sites in other Ig subtypes are understood in the art, and can be readily determined based on, e.g., the alignments provided in FIGS. 2B and 2C of U.S. Patent Publication No. 2009/0012272.

Even when cysteine residues are removed, the single chain Fc domains can still form a dimeric Fe domain that is held together non-covalently. A native Fe can be modified to make it more compatible with a selected host cell. For example, one can remove the PA sequence near the N-terminus of a typical native Fc, which may be recognized by a digestive enzyme in E. coli such as proline iminopeptidase. One can also add an N-terminal methionine residue, especially when the molecule is expressed recombinantly in a bacterial cell such as E. coli. The Fe domain of SEQ ID NO: 599 as described in U.S. Patent Publication No. 2009/0012272 is one such Fe variant. A portion of the N-terminus of a native Fe can be removed to prevent N-terminal heterogeneity when expressed in a selected host cell. For this purpose, one can delete any of the first 20 amino acid residues at the N-terminus, particularly those at positions 1, 2, 3, 4 and 5. One or more glycosylation sites can be removed. Residues that are typically glycosylated (e.g., asparagine) may confer cytolytic response. Such residues can be deleted or substituted with unglycosylated residues (e.g., alanine). Sites involved in interaction with complement, such as the Clq binding site, can be removed. For example, one can delete or substitute the EKK sequence of human IgG 1. Complement recruitment may not be advantageous for the molecules of this invention and so may be avoided with such an Fc variant. Sites that affect binding to Fe receptors other than a salvage receptor can be removed. A native Fc may have sites for interaction with certain white blood cells that are not required for the fusion molecules of the present invention and so can be removed. The ADCC site can be removed. ADCC sites are known in the art; see, for example, Molec. Immunol. 29 (5): 633-9 (1992) with regard to ADCC sites in IgG1. These sites, as well, are not required for a subject fusion polypeptide and so can be removed. When the native Fc is derived from a non-human antibody, the native Fc can be humanized. To humanize a native Fe, one can substitute selected residues in the non-human native Fe with residues that are normally found in human native Fc. Techniques for antibody humanization are well known in the art.

Exemplary suitable Fc variants include the following. In SEQ ID NO: 599 (as described in U.S. Patent Publication No. 2009/0012272; and as depicted in FIG. 5; SEQ ID NO:8) the leucine at position 15 may be substituted with glutamate; the glutamate at position 99, with alanine; and the lysines at positions 101 and 103, with alanines. In addition, one or more tyrosine residues can be replaced by phenyalanine residues.

Antibody fragments” comprise a portion of an intact antibody, for example the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 (1995)); single-chain antibody molecules; and multispecific antibodies formed from antibody fragments.

In some embodiments, a subject DDX3X-binding HCV core fusion polypeptide comprises an Fc domain polypeptide as the heterologous polypeptide, and is further modified with one or more water-soluble polymers. See, e.g., U.S. Patent Publication No. 2008/0254020.

In some embodiments, a subject DDX3X-binding HCV core fusion polypeptide is of the following formula: NH₂—X₁-(A)_{y}-X₂-B-X₃—COOH, where A is a DDX3X-binding HCV core polypeptide, and where y is an integer from 1 to about 5; B is a heterologous polypeptide; X₁, if present, is a peptide of from about 1 to about 50 amino acids in length; X₂, if present, is a linker peptide of from about 1 to about 50 amino acids in length; and X₃, if present, is a peptide of from about 1 to about 50 amino acids in length. In some embodiments, a subject DDX3X-binding HCV core fusion polypeptide is of the following formula: NH₂—X₁-B-X₂-(A)_{y}-X₃—COOH, where A is a DDX3X-binding HCV core polypeptide; B is a heterologous polypeptide; X₁, if present, is a peptide of from about 1 to about 50 amino acids in length; X₂, if present, is a linker peptide of from about 1 to about 50 amino acids in length; and X₃, if present, is a peptide of from about 1 to about 50 amino acids in length. Thus, in some embodiments, a subject DDX3X-binding HCV core fusion polypeptide comprises a DDX3X-binding HCV core polypeptide and a heterologous polypeptide fused, either directly or via a linker, to the amino terminus of the DDX3X-binding HCV core polypeptide. In other embodiments, a subject DDX3X-binding HCV core fusion polypeptide comprises a DDX3X-binding HCV core polypeptide and a heterologous polypeptide fused, either directly or via a linker, to the carboxyl-terminus of the DDX3X-binding HCV core polypeptide.

As indicated above, the DDX3X-binding HCV polypeptide can be present in multiple copies in a subject DDX3X-binding HCV core fusion polypeptide. Thus, for example, in some embodiments, a subject DDX3X-binding HCV core fusion polypeptide comprises two, three, four, or five copies of a DDX3X-binding HCV core polypeptide, which copies can be in tandem, either directly or separated by a linked.

A linker peptide, if present, can have a length of from about 5 amino acids to about 40 amino acids. The linker peptide may have any of a variety of amino acid sequences. Proteins can be joined by a spacer peptide, generally of a flexible nature, although other chemical linkages are not excluded. Suitable linker peptides can have a length of between about 5 and about 40 amino acids in length, e.g., between about 5 and about 25 amino acids in length. These linkers are generally produced by using synthetic, linker-encoding oligonucleotides to couple the proteins. Peptide linkers with a degree of flexibility are suitable for use, e.g., linkers having a relatively high proportion of glycine and/or serine residues. The linking peptides may have virtually any amino acid sequence. The use of small amino acids, such as glycine, serine, and alanine, are of use in creating a flexible peptide. The creation of such sequences is routine to those of skill in the art. Exemplary linker peptides include peptides comprising one of the following amino acid sequences: 1) GSGSGENLYFAGGGSGGSGS (SEQ ID NO:25); 2) GGGSGGSGSGSGSGSG (SEQ ID NO:26); and 3) GSGSGASGASGSGGGSG (SEQ ID NO:27).

In some embodiments, the linker peptide comprises a proteolytic cleavage site. Proteolytic cleavage sites are known to those skilled in the art; a wide variety are known and have been described amply in the literature, including, e.g., Handbook of Proteolytic Enzymes (1998) A J Barrett, N D Rawlings, and J F Woessner, eds., Academic Press. Proteolytic cleavage sites include, but are not limited to, an enterokinase cleavage site: (Asp)₄Lys; a factor Xa cleavage site: Ile-Glu-Gly-Arg; a thrombin cleavage site, e.g., Leu-Val-Pro-Arg-Gly-Ser; a renin cleavage site, e.g., His-Pro-Phe-His-Leu-Val-Ile-His; a collagenase cleavage site, e.g., X-Gly-Pro (where X is any amino acid); a trypsin cleavage site, e.g., Arg-Lys; a viral protease cleavage site, such as a viral 2A or 3C protease cleavage site, including, but not limited to, a protease 2A cleavage site from a picornavirus (see, e.g., Sommergruber et al. (1994) Virol. 198:741-745), a Hepatitis A virus 3C cleavage site (see, e.g., Schultheiss et al. (1995) J. Virol. 69:1727-1733), human rhinovirus 2A protease cleavage site (see, e.g., Wang et al. (1997) Biochem. Biophys. Res. Comm. 235:562-566), and a picornavirus 3 protease cleavage site (see, e.g., Walker et al. (1994) Biotechnol. 12:601-605.

A subject DDX3X-binding HCV core fusion polypeptide will in some embodiments further comprise one or more non-peptide modifications. Suitable non-peptide modifications include covalent modification with a polymer such as polyethylene glycols, copolymers of ethylene glycol/propylene glycol, polyvinyl alcohol, carboxymethylcellulose, polyvinyl pyrrolidone, poly-1,3-dioxolane, poly-1,3,6-trioxane, ethylene/maleic anhydride copolymer, polyaminoacids (either homopolymers or random copolymers), and dextran.

### Peptide Variants

A subject DDX3X-binding HCV core fusion polypeptide can comprise a “derivative” of a DDX3X-binding HCV core polypeptide, where a “derivative” of a DDX3X-binding HCV core polypeptide includes modifications other than, or in addition to, insertions, deletions, or substitutions of amino acid residues. In some embodiments, the modifications are covalent in nature, and include for example, chemical bonding with polymers, lipids, other organic and inorganic moieties. A subject derivative (e.g., a derivative a DDX3X-binding HCV core polypeptide) of can be prepared to increase circulating half-life (e.g., to increase in vivo half-life and/or serum half-life; to improve targeting capacity to desired cells, tissues, or organs; to improve the solubility or absorption; or to eliminate or attenuate any undesirable side-effect. Exemplary derivatives include DDX3X-binding HCV core polypeptides in which: 1. A DDX3X-binding HCV core polypeptide or some portion thereof is cyclic. For example, a DDX3X-binding HCV core polypeptide can be modified to contain two or more Cys residues (e.g., in the linker), which could cyclize by disulfide bond formation; 2. A DDX3X-binding HCV core polypeptide is cross-linked or is rendered capable of cross-linking between molecules. For example, a DDX3X-binding HCV core polypeptide can be modified to contain one Cys residue and thereby be able to form an intermolecular disulfide bond with a like molecule. A DDX3X-binding HCV core polypeptide may also be cross-linked through its C-terminus.

Peptides (e.g., a DDX3X-binding HCV core peptide, a DDX3X-binding HCV core polypeptide-containing fusion or a stabilized DDX3X-binding HCV core polypeptide fragment) can include naturally-occurring and non-naturally occurring amino acids. Peptides may comprise D-amino acids, a combination of D- and L-amino acids, and various “designer” amino acids (e.g., β-methyl amino acids, Cα-methyl amino acids, and Nα-methyl amino acids, etc.) to convey special properties to peptides. Additionally, peptide may be a cyclic peptide. Peptides may include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics may be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analog; amide bond isostere; tretrazol; and the like.

A peptide may be a depsipeptide, which may be a linear or a cyclic depsipeptide. Kuisle et al. (1999) Tet. Letters 40:1203-1206. “Depsipeptides” are compounds containing a sequence of at least two alpha-amino acids and at least one alpha-hydroxy carboxylic acid, which are bound through at least one normal peptide link and ester links, derived from the hydroxy carboxylic acids, where “linear depsipeptides” may comprise rings formed through S—S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. “Cyclic depsipeptides” are peptides containing at least one ring formed only through peptide or ester links, derived from hydroxy carboxylic acids.

Peptides may be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art.

The term “bicyclic” refers to a peptide in which there exists two ring closures. The ring closures are formed by covalent linkages between amino acids in the peptide. A covalent linkage between two nonadjacent amino acids constitutes a ring closure, as does a second covalent linkage between a pair of adjacent amino acids which are already linked by a covalent peptide linkage. The covalent linkages forming the ring closures may be amide linkages, i.e., the linkage formed between a free amino on one amino acid and a free carboxyl of a second amino acid, or linkages formed between the side chains or “R” groups of amino acids in the peptides. Thus, bicyclic peptides may be “true” bicyclic peptides, i.e., peptides cyclized by the formation of a peptide bond between the N-terminus and the C-terminus of the peptide, or they may be “depsi-bicyclic” peptides, i.e., peptides in which the terminal amino acids are covalently linked through their side chain moieties.

A desamino or descarboxy residue can be incorporated at the terminii of the peptide, so that there is no terminal amino or carboxyl group, to decrease susceptibility to proteases or to restrict the conformation of the peptide. C-terminal functional groups include amide, amide lower alkyl, amide di(lower alkyl), lower alkoxy, hydroxy, and carboxy, and the lower ester derivatives thereof, and the pharmaceutically acceptable salts thereof.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Such hydrophilic polymers can have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S., Bioconjugate Chem., 6:150-165 (1995); Monfardini, C, et al., Bioconjugate Chem., 6:62-69 (1995); U.S. Pat. Nos. 4,640,835; 4,496,689; 4,301,144; 4,670,417; 4,791,192; 4,179,337 or WO 95/34326.

Peptide aptamers are peptides or small polypeptides that act as dominant inhibitors of protein function. Peptide aptamers specifically bind to target proteins, blocking their function ability. Kolonin and Finley, PNAS (1998) 95:14266-14271. Due to the highly selective nature of peptide aptamers, they may be used not only to target a specific protein, but also to target specific functions of a given protein (e.g. a protein binding function). Further, peptide aptamers may be expressed in a controlled fashion by use of promoters which regulate expression in a temporal, spatial or inducible manner.

Peptide aptamers that bind with high affinity and specificity to a target protein may be isolated by a variety of techniques known in the art. Peptide aptamers can be isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

In a subject polypeptide, one or more peptidyl [—C(O)NR—] linkages (bonds) can be replaced by a non-peptidyl linkage. Exemplary non-peptidyl linkages are —CH₂-carbamate [—CH₂—OC(O)NR—], phosphonate, —CH₂-sulfonamide [—CH₂—S(O)₂NR—], urea [—NHC(O)NH—], CH₂-secondary amine, and alkylated peptide [—C(O)NR⁶— wherein R⁶ is lower alkyl]. The N-terminus of a subject polypeptide can be derivatized. For example, the N-terminus may be acylated or modified to a substituted amine. Exemplary N-terminal derivative groups include —NRR¹ (other than —NH₂), —NRC(O)R¹, —NRC(O)OR¹, —NRS(O)₂R¹—NHC(O)NHR¹, succinimide, or benzyloxycarbonyl-NH—(CBZ-NH—), wherein R and R.sup.1 are each independently hydrogen or lower alkyl and wherein the phenyl ring may be substituted with 1 to 3 substituents selected from the group consisting of C₁-C₄ alkyl, C₁1-C₄ alkoxy, chloro, and bromo. The free C-terminus can be derivatized. For example, the C-terminus is esterified or amidated. For example, one can use methods described in the art to add (NH—CH₂—CH₂—NH₂)₂ to compounds of this invention. Likewise, one can use methods described in the art to add —NH₂ to compounds of this invention. Exemplary C-terminal derivative groups include, for example, —C(O)R² wherein R² is lower alkoxy or —NR³R⁴ wherein R³ and R⁴ are independently hydrogen or C₁-C₈ alkyl (e.g., C₁-C₄). A disulfide bond can be replaced with another, more stable, cross-linking moiety (e.g., an alkylene). See, e.g., Bhatnagar et al. (1996), J. Med. Chem. 39: 3814-9; Alberts et al. (1993) Thirteenth Am. Pep. Symp., 357-9. One or more individual amino acid residues can be modified. Various derivatizing agents are known to react specifically with selected side chains or terminal residues.

Lysinyl residues and amino terminal residues can be reacted with succinic or other carboxylic acid anhydrides, which reverse the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues can be modified by reaction with any one or combination of several conventional reagents, including phenylglyoxal, 2,3-butanedione, 1,2-cyclohexanedione, and ninhydrin. Derivatization of arginyl residues can require that the reaction be performed in alkaline conditions because of the high pKa of the guanidine functional group. Furthermore, these reagents may react with the groups of lysine as well as the arginine epsilon-amino group.

Specific modification of tyrosyl residues has been studied extensively, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl sidechain groups (aspartyl or glutamyl) can be selectively modified by reaction with carbodiimides (R′—N═C═N—R′) such as 1-cyclohexyl-3-(2-morpholinyl-(4-ethyl)carbodiimide or 1-ethyl-3-(4-azonia-4,4-dimethylpentyl)carbodiimide. Furthermore, aspartyl and glutamyl residues may be converted to asparaginyl and glutaminyl residues by reaction with ammonium ions.

Glutaminyl and asparaginyl residues can be deamidated to the corresponding glutamyl and aspartyl residues. Alternatively, these residues are deamidated under mildly acidic conditions.

Cysteinyl residues can be replaced by amino acid residues or other moieties either to eliminate disulfide bonding or, conversely, to stabilize cross-linking. See, e.g., Bhatnagar et al. (1996), J. Med. Chem. 39: 3814-9.

Derivatization with bifunctional agents is useful for cross-linking the peptides or their functional derivatives to a water-insoluble support matrix or to other macromolecular vehicles. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) can be incorporated into a subject polypeptide, and can be glycosylated by a cell during recombinant production of the polypeptide (e.g., in mammalian cells such as CHO, BHK, COS). However, such sites may further be glycosylated by synthetic or semi-synthetic procedures known in the art.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), pp. 79-86 (1983).

Such derivatized moieties can improve one or more characteristics including solubility, absorption, biological half life, and the like of a subject polypeptide. Alternatively, derivatized moieties can result in compounds that have the same, or essentially the same, characteristics and/or properties of the compound that is not derivatized. The moieties can alternatively eliminate or attenuate any undesirable side effect of the compounds and the like.

Also provided are related peptides within the understanding of those with skill in the art, such as chemical mimetics, organomimetics, or peptidomimetics. As used herein, the terms “mimetic,” “peptide mimetic,” “peptidomimetic,” “organomimetic,” and “chemical mimetic” are intended to encompass peptide derivatives, peptide analogs, and chemical compounds having an arrangement of atoms in a three-dimensional orientation that is equivalent to that of a subject polypeptide. It will be understood that the phrase “equivalent to” as used herein is intended to encompass peptides having substitution(s) of certain atoms, or chemical moieties in said peptide, having bond lengths, bond angles, and arrangements in the mimetic peptide that produce the same or sufficiently similar arrangement or orientation of said atoms and moieties to have the biological function of a subject polypeptide (e.g., a DDX3X-binding HCV core polypeptide fragment). In a subject peptide mimetic, the three-dimensional arrangement of the chemical constituents is structurally and/or functionally equivalent to the three-dimensional arrangement of the peptide backbone and component amino acid sidechains in the peptide, resulting in such peptido-, organo-, and chemical mimetics of the peptides of the invention having substantial biological activity. These terms are used according to the understanding in the art, as illustrated, for example, by Fauchere, (Adv. Drug Res. 15:29, 1986); Veber & Freidinger, (TINS p. 392, 1985); and Evans, et al., (J. Med. Chem. 30:1229, 1987), incorporated herein by reference.

### Further Modifications

A subject DDX3X-binding HCV core fusion protein can be modified to include one or more of a non-proteinaceous polymer (e.g., a water-soluble polymer), a lipophilic compound, and an oligosaccharide, as described below.

### Stabilized DDX3X-Binding HCV Core Polypeptide Fragment

The present disclosure also provides a stabilized DDX3X-binding HCV core polypeptide fragment that comprises an amino acid sequence having at least about 75%, at least about 80%, at least about 85%, at least about 90%, at least about 95%, at least about 98%, at least about 99%, or 100%, amino acid sequence identity to a contiguous stretch of from about 5 amino acids to about 150 amino acids of any one of the amino acid sequences depicted in FIG. 4, where the DDX3X-binding HCV core polypeptide fragment includes amino acids 34-36 of any one of the amino acid sequences depicted in FIG. 4, and where the DDX3X-binding HCV core polypeptide fragment further comprises a modification that provides for one or more of enhanced serum half-life, improved in vitro and/or in vivo stability (e.g., increased in vivo and/or serum half-life), and improved in vitro and/or in vivo solubility. In these embodiments, the stabilized DDX3X-binding HCV core polypeptide fragment has a length of from about 5 amino acids to about 150 amino acids, e.g., from about 5 aa to about 10 aa, from about 10 aa to about 25 aa, from about 25 aa to about 50 aa, from about 50 aa to about 100 aa, or from about 100 aa to about 150 aa. Suitable exemplary modifications include, but are not limited to, covalent modification with a non-proteinaceous polymer; covalent modification with a water-soluble polymer; covalent modification with a lipophilic compound; addition of a carbohydrate moiety; addition of an oligosaccharide; and the like.

Suitable water-soluble polymers include, but are not limited to, polyethylene glycols, copolymers of ethylene glycol/propylene glycol, polyvinyl alcohol, carboxymethylcellulose, polyvinyl pyrrolidone, poly-1,3-dioxolane, poly-1,3,6-trioxane, ethylene/maleic anhydride copolymer, polyaminoacids (either homopolymers or random copolymers), and dextran.

In some embodiments, the polymer is a hydrophilic polymer. In some embodiments, the polymer is a hydrophilic synthetic polymer. A “synthetic” polymer is a polymer not normally found in nature. However, polymers which exist in nature and are produced by recombinant or in vitro methods are useful, as are polymers which are isolated from native sources. Suitable hydrophilic polyvinyl polymers include, e.g., polyvinylalcohol and polyvinylpyrrolidone.

Exemplary suitable polymers are polyalkylene ethers such as polyethylene glycol (PEG); polyelkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon. In some embodiments, the polymer prior to cross-linking is water soluble; in other embodiments, the polymer prior to cross-linking is not water soluble. In many embodiments, the final conjugate is water soluble. In addition, the polymer should not be highly immunogenic in the conjugate form, nor should it possess viscosity that is incompatible with intravenous infusion or injection if it is intended to be administered by such routes.

In some embodiments, the polymer contains only a single group which is reactive. However, it is within the scope herein to optimize reaction conditions to reduce cross-linking, or to purify the reaction products through gel filtration or chromatographic sieves to recover substantially homogenous derivatives.

The molecular weight of the polymer can range from about 100 to 500,000 Daltons, or from about 1,000 to 20,000 Daltons. The molecular weight chosen will depend, e.g., upon the nature of the polymer and the degree of substitution. In general, the greater the hydrophilicity of the polymer and the greater the degree of substitution, the lower the molecular weight that can be employed. Optimal molecular weights will be determined by routine experimentation.

The polymer can be covalently linked to the DDX3X-binding HCV core polypeptide fragment though a multifunctional crosslinking agent which reacts with the polymer and one or more amino acid or sugar residues of the DDX3X-binding HCV core polypeptide fragment to be linked.

Covalent binding to amino groups is accomplished by known chemistries based upon cyanuric chloride, carbonyl diimidazole, aldehyde reactive groups (PEG alkoxide plus diethyl acetal of bromoacetaldehyde; PEG plus DMSO and acetic anhydride, or PEG chloride plus the phenoxide of 4-hydroxybenzaldehyde, succinimidyl active esters, activated dithiocarbonate PEG, 2,4,5-trichlorophenylcloroformate or P-nitrophenylcloroformate activated PEG). Carboxyl groups are derivatized by coupling PEG-amine using carbodiimide.

The polymer can bear a group which is directly reactive with an amino acid side chain, or the N- or C-terminus of the DDX3X-binding HCV core polypeptide fragment linked, or which is reactive with the multifunctional cross-linking agent.

“Water soluble” in reference to the starting polymer means that the polymer or its reactive intermediate used for conjugation is sufficiently water soluble to participate in a derivatization reaction or cross-linking reaction. “Water soluble” in reference to the polymer conjugate (e.g., polymer-DDX3X-binding HCV core polypeptide fragment conjugate) means that the conjugate is soluble in physiological fluids such as blood.

The degree of substitution with such a polymer will vary depending upon the number of reactive sites on the DDX3X-binding HCV core polypeptide fragment, the molecular weight, hydrophilicity and other characteristics of the polymer, and the like. In general, the conjugate contains about from 1 to 10 polymer molecules, while any heterologous sequence may be substituted with an essentially unlimited number of polymer molecules so long as the desired activity is not significantly adversely affected. The optimal degree of cross-linking is easily determined by an experimental matrix in which the time, temperature and other reaction conditions are varied to change the degree of substitution, after which the ability of the conjugates to function in the desired fashion is determined.

A subject conjugate (e.g., a subject polymer-DDX3X-binding HCV core polypeptide fragment conjugate) can be separated from the unreacted starting materials by gel filtration. Heterologous species of the conjugates are purified from one another in the same fashion. The polymer also may be water-insoluble, as a hydrophilic gel.

As one non-limiting example, in some embodiments, a subject stabilized DDX3X-binding HCV core polypeptide fragment comprises amino acids 34-36 of any one of the amino acid sequences depicted in FIG. 4, has a length of from about 5 amino acids to about 150 amino acids, and is chemically modified with a single 20 kDa polyethylene glycol (PEG) polymer at the N-terminus of the DDX3X-binding HCV core polypeptide fragment.

In some embodiments, a subject stabilized DDX3X-binding HCV core polypeptide fragment comprises a DDX3X-binding HCV core polypeptide fragment conjugated to a lipophilic compound. For conjugation to a lipophilic compound the following polypeptide groups may function as attachment groups: the N-terminus or C-terminus of the polypeptide, the hydroxyl groups of the amino acid residues Ser, Thr or Tyr, the epsilon-amino group of Lys, the SH group of Cys or the carboxyl group of Asp and Glu. The polypeptide and the lipophilic compound may be conjugated to each other either directly or by use of a linker. The lipophilic compound may be a natural compound such as a saturated or unsaturated fatty acid, a fatty acid diketone, a terpene, a prostaglandin, a vitamin, a carotenoid or steroid, or a synthetic compound such as a carbon acid, an alcohol, an amine and sulphonic acid with one or more alkyl, aryl, alkenyl or other multiple unsaturated compounds. The conjugation between the polypeptide and the lipophilic compound, optionally through a linker may be done according to methods known in the art, e.g. as described by Bodanszky in Peptide Synthesis, John Wiley, New York, 1976 and in WO 96/12505.

It should be noted that any of the above-noted modifications can be made to a subject DDX3X-binding HCV core fusion protein. Thus, for example, a subject DDX3X-binding HCV core fusion protein can be modified to include one or more of a non-proteinaceous polymer (e.g., a water-soluble polymer), a lipophilic compound, and an oligosaccharide.

### Nucleic Acids

The present disclosure provides a nucleic acid comprising a nucleotide sequence encoding a recombinant polypeptide comprising: i) a DDX3X-interacting HCV core fragment; and ii) a heterologous polypeptide. Thus, the present disclosure provides a nucleic acid comprising a nucleotide sequence encoding a subject DDX3X-binding HCV core fusion polypeptide. The present disclosure further provides compositions, including pharmaceutical compositions, comprising a subject nucleic acid. The present disclosure further provides a recombinant expression vector comprising a subject nucleic acid. The present disclosure further provides host cells (e.g., isolated host cells) genetically modified with a subject nucleic acid or a subject recombinant expression vector.

In some embodiments, a subject nucleic acid provides for production of a subject DDX3X-binding HCV core fusion polypeptide, either in vitro or in vivo.

A subject DDX3X-binding HCV core fusion polypeptide nucleic acid can be a recombinant vector, e.g., a cloning vector or an expression vector comprising a nucleotide sequence encoding a subject DDX3X-binding HCV core fusion polypeptide. Cloning vectors generally provide for propagation of a nucleic acid; expression vectors generally provide for production of an encoded protein in a living cell and/or in an in vitro cell-free transcription/translation system. Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, a lentivirus, and the like), P1-based artificial chromosomes, yeast plasmids, yeast artificial chromosomes, and any other vectors specific for specific hosts of interest.

The expression vector will provide a transcriptional and translational initiation region, which may be inducible or constitutive, where the coding region is operably linked under the transcriptional control of the transcriptional initiation region, and a transcriptional and translational termination region. For generating a genetically modified host cell (as described below) comprising one or more heterologous nucleic acids comprising nucleotide sequences encoding a subject DDX3X-binding HCV core fusion polypeptide is inserted into an expression vector, and the expression vector is introduced into a host cell. Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Suitable expression vectors include, but are not limited to, viral vectors (e.g. viral vectors based on vaccinia virus; poliovirus; adenovirus (see, e.g., Li et al., Invest Opthalmol V is Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641648, 1999; Ali et al., Hum Mol Genet. 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; a salicylate promoter; in vivo regulated promoters, such as an ssaG promoter or a related promoter (see, e.g., U.S. Patent Publication No. 20040131637), a pagC promoter (Pulkkinen and Miller, J. Bacteriol., 1991: 173(1): 86-93; Alpuche-Aranda et al., PNAS, 1992; 89(21): 10079-83), a nirB promoter (Harborne et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter; and the like.

Non-limiting examples of suitable eukaryotic promoters (promoters functional in a eukaryotic cell) include cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present invention further provides host cells (e.g., isolated host cells) genetically modified with a subject nucleic acid.

Suitable host cells include primary cells and immortalized cell lines. Suitable host cells include eukaryotic cells, including mammalian cells, amphibian cells, plant cells, insect cells, and yeast cells.

Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some embodiments, the mammalian cell is one that can be infected with HCV. In some embodiments, the mammalian cell is a mammalian liver cell line. In some embodiments, the mammalian cell is a human liver cell line. In some embodiments, the mammalian cell is a Huh-7 cell. In some embodiments, the mammalian cell is one that comprises a molecular clone of HCV. In some embodiments, the mammalian cell is one that comprises an infectious molecular clone of HCV-N strain as described in Ikeda et al. (2002) J. Virol. 76:2997.

### Compositions

The present invention provides a composition comprising a subject nucleic acid, and compositions comprising a subject recombinant expression vector. A subject composition can comprise, in addition to a subject nucleic acid or a subject recombinant expression vector, one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; glycerol; and the like.

### Treatment Methods

The present disclosure provides methods of treating an HCV infection; and methods of treating complications or sequelae of an HCV infection, e.g., liver fibrosis. In some embodiments, a subject treatment method involves administering to an individual in need thereof an effective amount of a subject nucleic acid (e.g., a subject expression vector). In other embodiments, a subject treatment method involves administering to an individual in need thereof an effective amount of a subject DDX3X-binding HCV core fusion polypeptide. In other embodiments, a subject treatment method involves administering to an individual in need thereof an effective amount of a subject stabilized DDX3X-binding HCV core polypeptide fragment. For simplicity, the term “subject active agent” is used below to refer collectively to a subject nucleic acid, a subject DDX3X-binding HCV core fusion polypeptide, and a stabilized DDX3X-binding HCV core polypeptide fragment.

A subject active agent (e.g., a subject nucleic acid, a subject DDX3X-binding HCV core fusion polypeptide, or a stabilized DDX3X-binding HCV core polypeptide fragment) reduces the number of HCV virions produced by an HCV-infected cell. For example, in some embodiments, contacting an HCV-infected cell with a subject active agent reduces the number of HCV virions produced by an HCV-infected cell by at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90, or more than 90%, compared to the number of HCV virions produced by the HCV-infected cell not contacted with the active agent.

In some embodiments, an effective amount of a subject active agent (e.g., a subject nucleic acid, a subject DDX3X-binding HCV core fusion polypeptide, or a stabilized DDX3X-binding HCV core polypeptide fragment) is an amount that, when administered alone (e.g., in monotherapy) in one or more doses, is effective to reduce viral load or achieve a sustained viral response to therapy. In some embodiments, an effective amount of a subject active agent is an amount that, when administered alone (e.g., in monotherapy) in multiple (e.g., two or more) doses, is effective to reduce viral load or achieve a sustained viral response to therapy. In some embodiments, an effective amount of a subject active agent is an amount that, when administered in one or more doses in combination therapy with at least one additional therapeutic agent, is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

In some embodiments, an effective amount of a subject active agent (e.g., a subject nucleic acid, a subject DDX3X-binding HCV core fusion polypeptide, or a stabilized DDX3X-binding HCV core polypeptide fragment) is an amount that, when administered to an individual in need thereof in one or more doses, or alone or in combination therapy, is effective to reduce HCV viral titers to undetectable levels, e.g., to about 1000 to about 5000, to about 500 to about 1000, or to about 100 to about 500 genome copies/mL serum. In some embodiments, an effective amount of an active agent, and optionally one or more additional antiviral agents, is an amount that is effective to reduce viral load to lower than 5000 genome copies/mL serum. In some embodiments, an effective amount of a subject active agent, and optionally one or more additional antiviral agents, is an amount that is effective to reduce viral load to lower than 1000 genome copies/mL serum. In some embodiments, an effective amount of a subject active agent, and optionally one or more additional antiviral agents, is an amount that is effective to reduce viral load to lower than 500 genome copies/mL serum. In some embodiments, an effective amount of a subject active agent, and optionally one or more additional antiviral agents, is an amount that is effective to reduce viral load to lower than 100 genome copies/mL serum.

In some embodiments, an effective amount of a subject active agent is an amount that, when administered to an individual in need thereof in one or more doses, or alone or in combination therapy, is effective to achieve a 1.5-log, a 2-log, a 2.5-log, a 3-log, a 3.5-log, a 4-log, a 4.5-log, or a 5-log reduction in HCV viral titer in the serum of the individual.

Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR(RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT), developed by Gen-Probe Inc. (San Diego) and Chiron Corporation, and sold by Chiron Corporation under the trade name Procleix®, which NAT simultaneously tests for the presence of HIV-1 and HCV. See, e.g., Vargo et al. (2002) Transfusion 42:876-885.

In some embodiments, an effective amount of a subject active agent is an amount that, when administered to an individual in need thereof in one or more doses, or alone or in combination therapy, is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a subject active agent, and optionally one or more additional antiviral agents, is an amount effective to reduce ALT levels to less than about 45 IU/ml serum.

In some embodiments, an effective amount of a subject active agent is an amount that, when administered to an individual in need thereof in one or more doses, or alone or in combination therapy, is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

### Liver Fibrosis

Liver fibrosis is a precursor to the complications associated with liver cirrhosis, such as portal hypertension, progressive liver insufficiency, and hepatocellular carcinoma. A reduction in liver fibrosis thus reduces the incidence of such complications. Accordingly, the present invention further provides methods of reducing the likelihood that an individual will develop complications associated with cirrhosis of the liver.

A therapeutically effective amount of a subject active agent that is administered as part of a subject treatment method is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., an enzyme-linked immunosorbent assay (ELISA), a radioimmunoassay (RIA), and the like, using antibody specific for a given serum marker.

In the context of treating liver fibrosis, an “effective amounts” of a subject active agent is an amount that, when administered in one or more doses, in monotherapy or combination therapy, is effective in reducing liver fibrosis or reduce the rate of progression of liver fibrosis; and/or that is effective in reducing the likelihood that an individual will develop liver fibrosis; and/or that is effective in reducing a parameter associated with liver fibrosis; and/or that is effective in reducing a disorder associated with cirrhosis of the liver.

Whether a subject treatment method is effective in reducing liver fibrosis can be determined by any of a number of well-established techniques for measuring liver fibrosis and liver function. Whether liver fibrosis is reduced is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. These methods are described in more detail below.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

In some embodiments, an effective amount of a subject active agent is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or in a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., an ELISA, a RIA, and the like, using antibody specific for a given serum marker.

Quantitative tests of functional liver reserve can also be used to assess the efficacy of a subject treatment. These include: indocyanine green clearance (ICG), galactose elimination capacity (GEC), aminopyrine breath test (ABT), antipyrine clearance, monoethylglycine-xylidide (MEG-X) clearance, and caffeine clearance.

Reduction in liver fibrosis increases liver function. Thus, the invention provides methods for increasing liver function, the method generally involving administering to an individual in need thereof an effective amount of a subject active agent. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

### Administering a Subject Nucleic Acid

In some embodiments, a subject treatment method involves administering to an individual in need thereof an effective amount of a subject nucleic acid.

Conventional viral and non-viral based nucleic acid delivery methods can be used to introduce a subject nucleic acid into mammalian cells or target tissues. Such methods can be used to administer a subject nucleic acid to cells in vitro or in vivo. In some embodiments, a subject nucleic acid is administered to an individual. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of nucleic acid delivery methods, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Suitable methods for non-viral delivery of a subject nucleic acid include lipofection, microinjection, ballistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those described in, e.g., WO 91/17424 and WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to an individual (in vivo) or they can be used to treat cells in vitro and the modified cells are administered to an individual (ex vivo). Conventional viral based systems for the delivery of a subject nucleic acid could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene delivery methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting long terminal repeats (LTRs) are sufficient for replication and packaging of the vectors, which are then used to integrate the therapeutic gene into the target cell to provide permanent transgene expression. Exemplary suitable retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of the encoded polypeptide (e.g., a subject DDX3X-binding HCV core fusion polypeptide) is desired, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo nucleic acid delivery procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

pLASN and MFG-S are examples are retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)); such vectors are suitable for use in a subject method. PA317/pLASN was the first therapeutic vector used in a gene delivery trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are suitable gene delivery systems based on the defective and nonpathogenic parvovirus adeno-associated type 2 virus. The vectors are derived from a plasmid that retains only the AAV 145 by inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Nucleic acids can be delivered in vivo by administration to an individual patient, e.g., by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., liver cells, tissue biopsy), followed by reimplantation of the cells into the patient, usually after selection for cells which have incorporated the vector.

Vectors (e.g., retroviruses, adenoviruses, liposomes, etc.) can be also administered directly to an individual for transduction of cells in vivo, for delivery of a subject nucleic acid. Alternatively, naked DNA can be administered. Administration is by any of the routes normally used for introducing a molecule into ultimate contact with blood or tissue cells (e.g., liver cells). Suitable methods of administering such nucleic acids are available and well known to those of skill in the art, and, although more than one route can be used to administer a particular composition, a particular route can often provide a more immediate and more effective reaction than another route.

### Administering a Polypeptide

In some embodiments, a subject treatment method involves administering an effective amount of a subject polypeptide, e.g., a subject DDX3X-binding HCV core fusion polypeptide, or a stabilized DDX3X-binding HCV core polypeptide fragment.

In some embodiments, a subject polypeptide is linked to, or formulated with, an agent or a delivery vehicle that facilitates translocation across a eukaryotic cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the h (hydrophobic) domain of signal peptides, was found to have similar cell membrane translocation characteristics (see, e.g., Lin et al., J. Biol. Chem. 270:1 4255-14258 (1995)).

Examples of peptide sequences which can be linked to a subject polypeptide, for facilitating uptake of the polypeptide into cells, include, but are not limited to: an 11 amino acid peptide of the tat protein of HIV; a 20 residue peptide sequence which corresponds to amino acids 84-103 of the p16 protein (see Fahraeus et al., Current Biology 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O'Hare, Cell 88:223-233 (1997)). Other suitable chemical moieties that provide enhanced cellular uptake may also be chemically (e.g., covalently) linked to a subject polypeptide. For example, nuclear localization signals may be appended to enhance uptake into the nuclear compartment of cells.

Toxin molecules also have the ability to transport polypeptides across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., J. Biol. Chem., 268:3334-3341 (1993); Perelle et al., Infect. Immun, 61:5147-5156 (1993); Stenmark et al., J. Cell Biol. 113:1025-1032 (1991); Donnelly et al., PNAS 90:3530-3534 (1993); Carbonetti et al., Abstr. Annu. Meet. Am. Soc. Microbiol. 95:295 (1995); Sebo et al., Infect. Immun 63:3851-3857 (1995); Klimpel et al., PNAS U.S.A. 89:10277-10281 (1992); and Novak et al., J. Biol. Chem. 267:17186-17193 1992)). Such peptides can be used to translocate a subject polypeptide across a cell membrane. A subject polypeptide can be conveniently fused to or derivatized with such sequences. As an example, the translocation sequence is provided as part of a fusion protein. Optionally, a linker can be used to link a subject polypeptide and the translocation sequence. Any suitable linker can be used, e.g., a peptide linker.

A subject polypeptide can also be introduced into an animal cell, e.g., a mammalian cell (e.g., an HCV-infected liver cell), via liposomes or liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., a subject polypeptide. The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

A suitable liposome can comprise a subject polypeptide and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer & Bangham, Biochim Biophys. Acta 443:629-634 (1976); Fraley, et al., PNAS 76:3348-3352 (1979); Hope et al., Biochim. Biophys. Acta 812:55-65 (1985); Mayer et al., Biochim Biophys.

Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

### Formulations, Dosages, and Routes of Administration

A subject active agent, and optionally one or more additional therapeutic agents, is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In some embodiments, a subject active agent (e.g., a subject DDX3X-HCV core fusion polypeptide; a subject stabilized DDX3X-HCV core polypeptide fragment) is formulated with a carrier. The carrier can include minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (e.g., less than about ten amino acid residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; nonionic surfactants such as polysorbates, poloxamers, or PEG; and/or neutral salts, e.g., NaCl, KCl, MgCl₂, CaCl.

In some embodiments, a subject active agent (e.g., a subject DDX3X-HCV core fusion polypeptide; a subject stabilized DDX3X-HCV core polypeptide fragment) is formulated in a sustained release carrier. Suitable examples include semipermeable polymer matrices in the form of shaped articles, e.g. suppositories, or microcapsules. Implantable sustained release matrices include copolymers of L-glutamic acid and gamma ethyl-L-glutamate (U. Sidman et al., 1983, “Biopolymers” 22(1): 547-556), poly (2-hydroxyethyl-methacrylate) (R. Langer et al., 1981, “J. Biomed. Mater. Res.” 15:167-277 and R. Langer, 1982, “Chem. Tech.” 12: 98-105), ethylene vinyl acetate (R. Langer et al., Id.), or poly-D-(−)-3-Hydroxybutyric acid (EP 133,988A). Sustained release compositions also include liposomally entrapped DDX3X-HCV core fusion polypeptide or stabilized DDX3X-HCV core polypeptide fragment. Liposomes containing a subject DDX3X-HCV core fusion polypeptide or a subject stabilized DDX3X-HCV core polypeptide fragment are prepared by methods known in the art. See, e.g., DE 3,218,121A; Epstein et al. 1985, “Proc. Natl. Acad. Sci. USA” 82: 3688-3692; Hwang et al., 1980, “Proc. Natl. Acad. Sci. USA” 77: 4030-4034; EP 52322A; EP 36676A; EP 88046A; EP 143949A; EP 142641A Japanese patent application 83-118008; U.S. Pat. Nos. 4,485,045 and 4,544,545; and EP 102,324A. In some embodiments, the liposomes are of the small (about 200-800 Angstroms) unilamelar type in which the lipid content is greater than about 30 mol. % cholesterol, the selected proportion being adjusted for the optimal rate of leakage of a subject active agent from the liposome.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In a subject treatment method, a subject active agent (and optionally one or more additional active agents) can be administered to an individual in need thereof using any convenient means capable of resulting in the desired therapeutic effect. Thus, the agents can be incorporated into a variety of formulations for therapeutic administration. More particularly, a subject active agent (and optionally one or more additional active agents) can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants and aerosols.

As such, administration of a subject active agent (and optionally one or more additional active agents) can be achieved in various ways, including oral, buccal, rectal, parenteral, intraperitoneal, intradermal, subcutaneous, intramuscular, transdermal, intratracheal, etc., administration. In some embodiments, two different routes of administration are used. As one non-limiting example, a ribavirin is administered orally; and a subject active agent is administered subcutaneously by injection.

For oral delivery of a subject active agent (and optionally one or more additional active agents), a subject formulation will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

As one non-limiting example of a suitable oral formulation, a subject active agent (and optionally one or more additional active agents) can be formulated together with one or more pharmaceutical excipients and coated with an enteric coating, as described in U.S. Pat. No. 6,346,269. For example, a solution comprising a subject active agent (and optionally one or more additional active agents) and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for the active agent include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™ and shellac.

Suitable oral formulations also include a subject active agent (and optionally one or more additional active agents) formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B. V.).

Suitable oral formulations also include a subject active agent (and optionally one or more additional active agents) formulated with any of the following: a carrier such as Emisphere® (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Subcutaneous administration of a subject active agent (and optionally one or more additional active agents) can be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, a subject active agent (and optionally one or more additional active agents) is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are used in some embodiments because of convenience in implantation and removal of the drug delivery device.

Drug release devices suitable for use in the invention can be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems will in some embodiments be used, due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In pharmaceutical dosage forms, a subject active agent (and optionally one or more additional active agents) is administered in the form of its pharmaceutically acceptable salts, or the active agent is used alone or in appropriate association, as well as in combination, with other pharmaceutically active compounds. The following methods and excipients are merely exemplary and are in no way limiting.

For oral preparations, a subject active agent (and optionally one or more additional active agents) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A subject active agent (and optionally one or more additional active agents) can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the active agents can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions can be provided wherein each dosage unit, for example, teaspoonful, tablespoonful, tablet or suppository, contains a predetermined amount of the composition containing one or more inhibitors. Similarly, unit dosage forms for injection or intravenous administration can comprise the active agent(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for a particular active agent depend on the particular agent employed and the effect to be achieved, and the pharmacodynamics associated with each agent in the host.

In connection with a subject treatment method, the present disclosure provides embodiments in which the therapeutic agent(s) is/are administered to an individual in need thereof by a controlled drug delivery device. In some embodiments, the therapeutic agent(s) is/are delivered to the patient substantially continuously or continuously by the controlled drug delivery device. Optionally, an implantable infusion pump is used to deliver the therapeutic agent(s) to the patient substantially continuously or continuously by subcutaneous infusion. In other embodiments, a therapeutic agent is administered to the patient so as to achieve and maintain a desired average daily serum concentration of the therapeutic agent at a substantially steady state for the duration of the monotherapy or combination therapy. Optionally, an implantable infusion pump is used to deliver the therapeutic agent to the patient by subcutaneous infusion so as to achieve and maintain a desired average daily serum concentration of the therapeutic agent at a substantially steady state for the duration of the therapeutic agent in monotherapy or combination therapy.

### Combination Therapy

In some embodiments, a subject treatment method involves administering: 1) a subject nucleic acid, a subject DDX3X-binding HCV core fusion polypeptide, or a subject stabilized DDX3X-binding HCV core polypeptide fragment; and 2) at least one additional therapeutic agent, in combined effective amounts to treat the HCV infection. Suitable additional therapeutic agents include agents suitable for treating an HCV infection, e.g., an interferon-alpha (IFN-α), a nucleoside analog, an HCV NS3 inhibitor, an HCV NS5B inhibitor, etc.

### Ribavirin

In some embodiments, the at least one additional suitable therapeutic agent includes ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin can be administered orally in capsule or tablet form, or in the same or different administration form and in the same or different route as a subject active agent. Of course, other types of administration of both medicaments, as they become available are contemplated, such as by nasal spray, transdermally, by suppository, by sustained release dosage form, etc. Any form of administration is suitable so long as the proper dosages are delivered without destroying the active ingredient.

Ribavirin can be administered in an amount ranging from about 400 mg to about 1200 mg, from about 600 mg to about 1000 mg, or from about 700 to about 900 mg per day. In some embodiments, ribavirin is administered throughout the entire course of therapy with a subject active agent.

### Levovirin

In some embodiments, the at least one additional suitable therapeutic agent includes levovirin. Levovirin is the L-enantiomer of ribavirin.

Levovirin has the following structure:

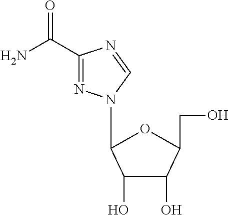

### Viramidine

In some embodiments, the at least one additional suitable therapeutic agent includes viramidine. Viramidine is a 3-carboxamidine derivative of ribavirin, and acts as aprodrug of ribavirin. It is efficiently converted to ribavirin by adenosine deaminases.

Viramidine has the following structure:

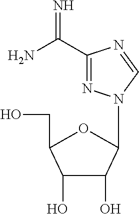

### Nucleoside Analogs

Nucleoside analogs that are suitable for use in a subject treatment method include, but are not limited to, ribavirin, levovirin, viramidine, isatoribine, an L-ribofuranosyl nucleoside as disclosed in U.S. Pat. No. 5,559,101 and encompassed by Formula I of U.S. Pat. No. 5,559,101 (e.g., 1-β-L-ribofuranosyluracil, 1-β-L-ribofuranosyl-5-fluorouracil, 1-β-L-ribofuranosylcytosine, 9-β-L-ribofuranosyladenine, 9-β-L-ribofuranosylhypoxanthine, 9-β-L-ribofuranosylguanine, 9-β-L-ribofuranosyl-6-thioguanine, 2-amino-α-L-ribofuran1[1′,2′:4,5]oxazoline, O²,O²-anhydro-1-α-L-ribofuranosyluracil, 1-α-L-ribofuranosyluracil, 1-(2,3,5-tri-O-benzoyl-α-ribofuranosyl)-4-thiouracil, 1-α-L-ribofuranosylcytosine, 1-α-L-ribofuranosyl-4-thiouracil, 1-α-L-ribofuranosyl-5-fluorouracil, 2-amino-β-L-arabinofurano[1′,2′:4,5]oxazoline, O²,O²-anhydro-β-L-arabinofuranosyluracil, 2′-deoxy-β-L-uridine, 3′5′-Di-O-benzoyl-2′ deoxy-4-thio β-L-uridine, 2′-deoxy-β-L-cytidine, 2′-deoxy-β-L-4-thiouridine, 2′-deoxy-β-L-thymidine, 2′-deoxy-β-L-5-fluorouridine, 2′,3′-dideoxy-β-L-uridine, 2′-deoxy-β-L-5-fluorouridine, and 2′-deoxy-β-L-inosine); a compound as disclosed in U.S. Pat. No. 6,423,695 and encompassed by Formula I of U.S. Pat. No. 6,423,695; a compound as disclosed in U.S. Patent Publication No. 2002/0058635, and encompassed by Formula 1 of U.S. Patent Publication No. 2002/0058635; a nucleoside analog as disclosed in WO 01/90121 A2 (Idenix); a nucleoside analog as disclosed in WO 02/069903 A2 (Biocryst Pharmaceuticals Inc.); a nucleoside analog as disclosed in WO 02/057287 A2 or WO 02/057425 A2 (both Merck/Isis); and the like.

### HCV NS3 Inhibitors

In some embodiments, the at least one additional suitable therapeutic agent includes HCV NS3 inhibitors. Suitable HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301 A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54^{th} Ann. Meeting AASLD); NS3 inhibitor SCH₆ (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); an NS3 protease inhibitor as disclosed in any one of U.S. Patent Publication Nos. 2003019067, 20030187018, 20030186895, 2007/0054842, and 2008/0019942; and the like.

Of particular interest in many embodiments are NS3 inhibitors that are specific NS3 inhibitors, e.g., NS3 inhibitors that inhibit NS3 serine protease activity and that do not show significant inhibitory activity against other serine proteases such as human leukocyte elastase, porcine pancreatic elastase, or bovine pancreatic chymotrypsin, or cysteine proteases such as human liver cathepsin B.

### NS5B Inhibitors

In some embodiments, the at least one additional suitable therapeutic agent includes NS5B inhibitors. Suitable HCV non-structural protein-5 (NS5; RNA-dependent RNA polymerase) inhibitors include, but are not limited to, a compound as disclosed in U.S. Pat. No. 6,479,508 (Boehringer-Ingelheim); a compound as disclosed in any of International Patent Application Nos. PCT/CA02/01127, PCT/CA02/01128, and PCT/CA02/01129, all filed on Jul. 18, 2002 by Boehringer Ingelheim; a compound as disclosed in U.S. Pat. No. 6,440,985 (ViroPharma); a compound as disclosed in WO 01/47883, e.g., JTK-003 (Japan Tobacco); a dinucleotide analog as disclosed in Zhong et al. (2003) Antimicrob. Agents Chemother. 47:2674-2681; a benzothiadiazine compound as disclosed in Dhanak et al. (2002) J. Biol. Chem. 277(41):38322-7; an NSSB inhibitor as disclosed in WO 02/100846 A1 or WO 02/100851 A2 (both Shire); an NSSB inhibitor as disclosed in WO 01/85172 A1 or WO 02/098424 A1 (both Glaxo SmithKline); an NSSB inhibitor as disclosed in WO 00/06529 or WO 02/06246 A1 (both Merck); an NSSB inhibitor as disclosed in WO 03/000254 (Japan Tobacco); an NSSB inhibitor as disclosed in EP 1 256,628 A2 (Agouron); JTK-002 (Japan Tobacco); JTK-109 (Japan Tobacco); and the like.

Of particular interest in many embodiments are NS5 inhibitors that are specific NS5 inhibitors, e.g., NS5 inhibitors that inhibit NS5 RNA-dependent RNA polymerase and that lack significant inhibitory effects toward other RNA dependent RNA polymerases and toward DNA dependent RNA polymerases.

### Interferon-Alpha

In some embodiments, the at least one additional suitable therapeutic agent includes an IFN-α. Any known IFN-α can be used in the instant invention. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated IFN-α, glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron-A interferon available from Schering Corporation, Kenilworth, N.J.; recombinant interferon alpha-2a such as Roferon interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Weliferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon Tradename.

The term “IFN-α” also encompasses consensus IFN-α. Consensus IFN-α (also referred to as “CIFN” and “IFN-con” and “consensus interferon”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., INFERGEN™), InterMune, Inc., Brisbane, Calif.). IFN-con₁ is the consensus interferon agent in the INFERGEN™ alfacon-1 product. The INFERGEN™ consensus interferon product is referred to herein by its brand name (INFERGEN™) or by its generic name (interferon alfacon-1). DNA sequences encoding IFN-con can be synthesized as described in the aforementioned patents or other standard methods.

Also suitable for use in a subject treatment method are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in a subject treatment method are gene-shuffled forms of IFN-α. See, e.g., Masci et al. (2003) Curr. Oncol. Rep. 5:108-113.

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boebringer Ingelheim, Ingelheim, Germany); and consensus interferon as defined by determination of a consensus sequence of naturally occurring interferon alphas (INFERGEN™, InterMune, Inc., Brisbane, Calif.).

### Subjects Suitable for Treatment

Individuals who are to be treated according to the methods of the invention include individuals who have been clinically diagnosed as infected with HCV. Individuals who are infected with HCV are identified as having HCV RNA in their blood, and/or having anti-HCV antibody in their serum.

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), e.g., a difficult to treat genotype such as HCV genotype 1, or particular HCV subtypes and quasispecies. In some embodiments, the individual is infected with HCV genotype 1. In some embodiments, the individual is infected with HCV genotype 1b.

Also of interest are HCV-positive individuals who exhibit severe fibrosis or early cirrhosis (non-decompensated, Child's-Pugh class A or less), or more advanced cirrhosis (decompensated, Child's-Pugh class B or C) due to chronic HCV infection. In particular embodiments of interest, HCV-positive individuals with stage 3 or 4 liver fibrosis according to the METAVIR scoring system are suitable for treatment with the methods of the present invention. In other embodiments, individuals suitable for treatment with the methods of the instant invention are patients with decompensated cirrhosis with clinical manifestations, including patients with far-advanced liver cirrhosis, including those awaiting liver transplantation. In still other embodiments, individuals suitable for treatment with the methods of the instant invention include patients with milder degrees of fibrosis including those with early fibrosis (stages 1 and 2 in the METAVIR, Ludwig, and Scheuer scoring systems; or stages 1, 2, or 3 in the Ishak scoring system.).

Individuals who are clinically diagnosed as infected with HCV include naive individuals (e.g., individuals not previously treated for HCV) and individuals who have failed prior treatment for HCV (“treatment failure” patients).

The term “treatment failure patients” (or “treatment failures”) as used herein generally refers to HCV-infected patients who failed to respond to previous therapy for HCV (referred to as “non-responders”) or who initially responded to previous therapy, but in whom the therapeutic response was not maintained (referred to as “relapsers”).

Patients for whom the therapy of the invention is of particular benefit include treatment failure patients, which include patients who failed to respond to previous HCV therapy (referred to as “non-responders”) or who initially responded to previous therapy, but in whom the therapeutic response was not maintained (referred to as “relapsers”). As non-limiting examples, individuals may have an HCV titer of at least about 10⁵, at least about 5×10⁵, or at least about 10⁶, genome copies of HCV per milliliter of serum.

Individuals who are to be treated according to the methods of the invention for treating an HCV infection include individuals who have been clinically diagnosed as infected with HCV. Individuals who are infected with HCV are identified as having HCV RNA in their blood, and/or having anti-HCV antibody in their serum.

In some embodiments, an individual to be treated according to a subject treatment method is an individual who has liver steatosis and who is HCV infected.

### Screening Methods

The present disclosure provides a method for identifying an agent that inhibits interaction of HCV core polypeptide with a DDX3X polypeptide. The methods generally involve contacting a test agent with an HCV core polypeptide and a DDX3X polypeptide; and determining the effect, if any, of the test agent on binding of the HCV core polypeptide to the DDX3X polypeptide. A test agent that inhibits binding of an HCV core polypeptide to a DDX3X polypeptide by at least about 10%, at least about 15%, at least about 20%, at least about 25%, at least about 30%, at least about 35%, at least about 40%, at least about 45%, at least about 50%, at least about 60%, at least about 70%, or at least about 80%, or more than 80%, compared to the binding of the HCV core polypeptide to the DDX3X polypeptide in the absence of the test agent, is considered a candidate agent for inhibiting HCV replication in a cell. Thus, a subject method provides for identifying a candidate agent for inhibiting HCV replication in a cell, and thus provides for identifying a candidate agent for treating an HCV infection in an individual.

A test agent can be further assessed for its ability to reduce HCV replication in a mammalian cell. For example, a test agent that has been determined to inhibit binding of HCV core to DDX3X can be further tested by contacting the agent with a cell in vitro, where the cell is infected with HCV or is genetically modified with an HCV clone; and the effect, if any, of the test agent on HCV replication can be determined. Methods for assessing HCV replication are known in the art; any such method can be used in connection with a subject screening method.

As used herein, the term “determining” refers to both quantitative and qualitative determinations and as such, the term “determining” is used interchangeably herein with “assaying,” “measuring,” and the like.

The terms “candidate agent,” “test agent,” “agent,” “substance,” and “compound” are used interchangeably herein. Candidate agents encompass numerous chemical classes, e.g., synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents can be small organic or inorganic compounds having a molecular weight of more than 50 and less than about 10,000 daltons, less than about 5,000 daltons, or less than about 2,500 daltons. Candidate agents can be organic compounds having a molecular weight in a range of from about 50 daltons to about 20,000 daltons, e.g., from about 50 daltons to about 100 daltons, from about 100 daltons to about 500 daltons, from about 500 daltons to about 1 kilodalton (kDa), from about 1 kDa to about 5 kDa, from about 5 kDa to about 10 kDa, from about 10 kDa to about 15 kDa, or from about 15 kDa to about 20 kDa.

Candidate agents can comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

Assays of the invention include controls, where suitable controls include a sample (e.g., a sample comprising a DDX3X polypeptide and a DDX3X-binding HCV core polypeptide, or a cell that synthesizes a DDX3X polypeptide and a DDX3X-binding HCV core polypeptide) in the absence of the test agent. Generally a plurality of assay mixtures is run in parallel with different agent concentrations to obtain a differential response to the various concentrations. Typically, one of these concentrations serves as a negative control, i.e. at zero concentration or below the level of detection.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Typically between 1 second and 1 hour will be sufficient.

Determining the effect, if any, of a test agent on binding between a DDX3X polypeptide and a DDX3X-binding HCV core polypeptide can be carried out using any of a variety of assays, including, but not limited to, immunological assays (e.g., enzyme-linked immunosorbent assays; radioimmunoassay; and the like); FRET-based assays; BRET-based assays; or any other assay that detects protein-protein binding.

In some embodiments, the screening method is carried out in vitro, in a cell-free assay. In some embodiments, the in vitro cell-free assay will employ an isolated DDX3X polypeptide and an isolated DDX3X-binding HCV core polypeptide. In some embodiments, the in vitro cell-free assay will employ a purified DDX3X polypeptide and purified DDX3X-binding HCV core polypeptide, where “purified” refers to free of contaminants or any other undesired components. Purified DDX3X polypeptide and purified DDX3X-binding HCV core polypeptide that is suitable for a subject screening method will in some embodiments be at least about 50% pure, at least about 60% pure, at least about 70% pure, at least about 75% pure, at least about 80% pure, at least about 85% pure, at least about 90% pure, at least about 95% pure, at least about 98% pure, at least about 99% pure, or greater than 99% pure.

A candidate agent is assessed for any cytotoxic activity (other than anti-proliferative activity) it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit cytotoxic activity are considered candidate agents.

In some embodiments, one or both of the DDX3X polypeptide and the DDX3X-binding HCV core polypeptide is detectably labeled (“tagged”). The terms “tag,” “detectable label,” and “detectable tag” are used interchangeably herein without limitation. In some embodiments, the tag is covalently bound to the attached component. By “tag,” “label,” “detectable label,” or “detectable tag” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is performed will depend on the label. Exemplary labels include, but are not limited to, fluorescent labels (e.g. a green fluorescent protein, a red fluorescent protein, a yellow fluorescent protein, etc.) and label enzymes.

Exemplary tags include, but are not limited to, an optically-detectable label, a partner of a binding pair, and a surface substrate binding molecule (or attachment tag). As will be evident to the skilled artisan, many molecules may find use as more than one type of tag, depending upon how the tag is used. In one embodiment, the tag or label as described below is incorporated into the polypeptide as a fusion protein.

The tag polypeptide can be, for example, an immunodetectable label (i.e., a polypeptide or other moiety which provides an epitope to which an anti-tag antibody can selectively bind), a polypeptide which serves as a ligand for binding to a receptor (e.g., to facilitate immobilization of the chimeric molecule on a substrate); an enzyme label (e.g., as described further below); or a fluorescent label (e.g., as described further below). Tag polypeptides provide for, for example, detection using an antibody against the tag polypeptide, and/or a ready means of isolating or purifying the tagged polypeptide (e.g., by affinity purification using an anti-tag antibody or another type of receptor-ligand matrix that binds to the tag). The production of tag-polypeptides by recombinant means is within the knowledge and skill in the art.

Production of immunodetectably-labeled proteins (e.g., use of FLAG, H is (e.g., polyhistidine), and the like, as a tag) is well known in the art and kits for such production are commercially available (for example, from Kodak and Sigma). See, e.g., Winston et al., Genes and Devel. 13:270-283 (1999), incorporated herein in its entirety, as well as product handbooks provided with the above-mentioned kits. Production of proteins having His-tags by recombinant means is well known, and kits for producing such proteins are commercially available. Such a kit and its use is described in the QIAexpress Handbook from Qiagen by Joanne Crowe et al., hereby expressly incorporated by reference.

Methods of producing polypeptides having an optically-detectable label are well known. An “optically detectable label” includes labels that are detectably due to inherent properties (e.g., a fluorescent label), or which can be reacted with a substrate or act as a substrate to provide an optically detectable (e.g., colored) reaction product (e.g., horse radish peroxidase).

By “fluorescent label” is meant any molecule that may be detected via its inherent fluorescent properties, which include fluorescence detectable upon excitation. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 2002 Molecular Probes Handbook, 9th Ed., by Richard P. Haugland, hereby expressly incorporated by reference.

Suitable fluorescent labels include, but are not limited to, green fluorescent protein (GFP; Chalfie, et al., Science 263(5148):802-805 (Feb. 11, 1994); and EGFP; Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), β-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558), a GFP from species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; U.S. Patent Publication No. 2002/0197676, or U.S. Patent Publication No. 2005/0032085; and the like.

In some instances, multiple fluorescent labels are employed. In one embodiment, at least two fluorescent labels are used which are members of a fluorescence resonance energy transfer (FRET) pair. FRET can be used to detect association/dissociation of a DDX3X polypeptide and a DDX3X-binding HCV core polypeptide; and the like. In general, such FRET pairs are used in in vitro assays.

FRET is phenomenon known in the art wherein excitation of one fluorescent dye is transferred to another without emission of a photon. A FRET pair consists of a donor fluorophore and an acceptor fluorophore (where the acceptor fluorophore may be a quencher molecule). The fluorescence emission spectrum of the donor and the fluorescence absorption spectrum of the acceptor must overlap, and the two molecules must be in close proximity The distance between donor and acceptor at which 50% of donors are deactivated (transfer energy to the acceptor) is defined by the Forster radius, which is typically 10-100 angstroms. Changes in the fluorescence emission spectrum comprising FRET pairs can be detected, indicating changes in the number of that are in close proximity (i.e., within 100 angstroms of each other). This will typically result from the binding or dissociation of two molecules, one of which is labeled with a FRET donor and the other of which is labeled with a FRET acceptor, wherein such binding brings the FRET pair in close proximity.

Binding of such molecules will result in an increased fluorescence emission of the acceptor and/or quenching of the fluorescence 15 emission of the donor. FRET pairs (donor/acceptor) useful in the invention include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5 and fluorescein/LC Red 705.

In another aspect of FRET, a fluorescent donor molecule and a nonfluorescent acceptor molecule (“quencher”) may be employed. In this application, fluorescent emission of the donor will increase when quencher is displaced from close proximity to the donor and fluorescent emission will decrease when the quencher is brought into close proximity to the donor. Useful quenchers include, but are not limited to, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL and fluorescein/QSY 7 dye.

The skilled artisan will appreciate that FRET and fluorescence quenching allow for monitoring of binding of labeled molecules over time, providing continuous information regarding the time course of binding reactions. Attachment of labels or other tags should not interfere with active groups on the interacting polypeptides Amino acids or other moieties can be added to the sequence of a protein, through means well known in the art and described herein, for the express purpose of providing a linker and/or point of attachment for a label. In one embodiment, one or more amino acids are added to the amino acid sequence of a polypeptide for attaching a tag thereto, with a fluorescent label being of particular interest.

In other embodiments, detection involves bioluminescence resonance energy transfer (BRET). BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. BRET assays may be performed by analyzing transfer between a bioluminescent donor protein and a fluorescent acceptor protein. Interaction between the donor and acceptor proteins can be monitored by a change in the ratio of light emitted by the bioluminescent and fluorescent proteins.

Alternatively, binding may be assayed by fluorescence anisotropy. Fluorescence anisotropy assays are amply described in the literature. See, e.g., Jameson and Sawyer (1995) Methods Enzymol. 246:283-300.

By “label enzyme” is meant an enzyme which may be reacted in the presence of a label enzyme substrate which produces a detectable product. Suitable label enzymes also include optically detectable labels (e.g., in the case of HRP). Suitable label enzymes for use in the present invention include but are not limited to, horseradish peroxidase (HRP), alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By “radioisotope” is meant any radioactive molecule. Suitable radioisotopes for use in the invention include, but are not limited to ¹⁴C, ³H, ³²P, ³³P, ³⁵S, ¹²⁵I, and ¹³¹I. The use of radioisotopes as labels is well known in the art.

In addition, labels may be indirectly detected, that is, the tag is a partner of a binding pair. By “partner of a binding pair” is meant one of a first and a second moiety, wherein said first and said second moiety have a specific binding affinity for each other. Suitable binding pairs for use in the invention include, but are not limited to, antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide (Hopp et al., BioTechnol, 6:1204-1210 (1988)); the KT3 epitope peptide (Martin et al., Science, 255:192-194 (1992)); tubulin epitope peptide (Skinner et al., J. Biol. Chem., 266: 15 163-15 166 (1991)); and the T7 gene 10 protein peptide tag (Lutz-Freyemuth et al., Proc. Natl. Acad. Sci. USA, a:6393-6397 (1990)) and the antibodies each thereto. Generally, in one embodiment, the smaller of the binding pair partners serves as the tag, as steric considerations in ubiquitin ligation may be important. As will be appreciated by those in the art, binding pair partners may be used in applications other than for labeling, such as immobilization of the protein on a substrate and other uses as described below.

As will be appreciated by those in the art, a partner of one binding pair may also be a partner of another binding pair. For example, an antigen (first moiety) may bind to a first antibody (second moiety) which may, in turn, be an antigen for a second antibody (third moiety). It will be further appreciated that such a circumstance allows indirect binding of a first moiety and a third moiety via an intermediary second moiety that is a binding pair partner to each. As will be appreciated by those in the art, a partner of a binding pair may comprise a label, as described above. It will further be appreciated that this allows for a tag to be indirectly labeled upon the binding of a binding partner comprising a label. Attaching a label to a tag which is a partner of a binding pair, as just described, is referred to herein as “indirect labeling.”

In one embodiment, the tag is surface substrate binding molecule. By “surface substrate binding molecule” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly- his-gly) tags and Nickel substrate; the Glutathione-S Transferase (GST) tag and its antibody substrate (available from Pierce Chemical); the influenza hemagglutinin (HA) tag polypeptide and its antibody 12CA5 substrate (Field et al., Mol. Cell. Biol., 8:2159-2165 (1988)); the c-myc tag and the 8F9, 3C7, 6E107 G4, B7 and 9E10 antibody substrates thereto (Evan et al., Molecular and Cellular Biol, 5:3610-3616 (1985)1; and the Herpes Simplex virus glycoprotein D (gD) tag and its antibody substrate (Paborsky et al., Protein Engineering, 3(6):547-553 (1990)). In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Production of antibody-embedded substrates is well known; see Slinkin et al., Bioconj, Chem. 2:342-348 (1991); Torchilin et al., supra; Trubetskoy et al., Bioconi. Chem. 33323-327 (1992); King et al., Cancer Res. 54:6176-6185 (1994); and Wilbur et al., Bioconjugate Chem. 5:220-235 (1994), and attachment of or production of proteins with antigens is described above. Calmodulin-embedded substrates are commercially available and production of proteins with CBP is described in Simcox et al., Strategies 8:40-43 (1995).

Where appropriate, functionalization of labels with chemically reactive groups such as thiols, amines, carboxyls, etc. is generally known in the art. In one embodiment, the tag is functionalized to facilitate covalent attachment.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see, e.g., chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known. Methods for labeling of proteins with radioisotopes are known in the art. For example, such methods are found in Ohta et al., Molec. Cell 3:535-541 (1999), which is hereby incorporated by reference in its entirety.

The covalent attachment of the tag may be either direct or via a linker. In one embodiment, the linker is a relatively short coupling moiety that is used to attach the molecules. A coupling moiety may be synthesized directly onto a component of the invention, ubiquitin for example, and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer.

### Cell-Based In Vitro Assays

As noted above, in some embodiments, a subject screening method is carried out in vitro, in a cell-based assay, where the DDX3X polypeptide and the DDX3X-binding HCV core polypeptide are present in a cell. In these embodiments, the cell is contacted with a test agent; and the effect, if any, of the test agent on binding of the DDX3X polypeptide to the DDX3X-binding HCV core polypeptide is determined. For these assays, a FRET- or BRET-based assay may be used. In some embodiments, the cell is genetically modified with one or more nucleic acids comprising nucleotide sequences encoding the DDX3X and DDX3X-binding HCV core polypeptides. The nucleic acids can be expression constructs which provide for production of DDX3X and DDX3X-binding HCV core polypeptides in the cell. Cells suitable for use with such assay methods are generally any higher eukaryotic cells in which DDX3X and DDX3X-binding HCV core polypeptides are produced or can be produced, e.g., cells which have been genetically modified to produce DDX3X and DDX3X-binding HCV core polypeptides polypeptides. In some embodiments, the host cells in the assays are mammalian cells. In some embodiments, the host cells in the assays are cells of a mammalian liver cell line. Suitable host cells include eukaryotic host cells that can be cultured in vitro, either in suspension or as adherent cells.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In general, the genetically modified cells can be produced using standard methods. Expression constructs comprising nucleotide sequences encoding DDX3X and DDX3X-binding HCV core polypeptides are introduced into the host cell using standard methods practiced by one with skill in the art. Where one or more recombinant polypeptides are to be introduced into the cell as a polynucleotides encoding the one or more polypeptides and an expression cassette, optionally carried on one or more transient expression vectors (e.g., the vector is maintained in an episomal manner by the cell), which comprise the polynucleotides encoding the desired polypeptides. Alternatively, or in addition, the one or more expression constructs encoding one or more polypeptides can be stably integrated into the cell line. In addition or alternatively, one or more of polynucleotides encoding one or more desired polypeptides can be stably integrated into the cell, while one or more other desired polypeptides expressed from one or more transient expression vectors. For example, a polynucleotide encoding a DDX3X polypeptide may be stably integrated in the cell line, while a polynucleotide encoding a DDX3X-binding HCV core polypeptide is expressed from one or more transient expression vectors. Likewise, a polynucleotide encoding a DDX3X-binding HCV core polypeptide may be stably integrated in the cell line, while a polynucleotide encoding a detectably labeled DDX3X polypeptide is expressed from a transient expression vector. Other variations and combinations of stably integrated vectors and transient expression vectors will be readily apparent to the skilled artisan upon reading the present disclosure.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Dominant Negative Inhibition of HCV Replication by Linking a Fragment of HCV Core Protein to the C-Terminus of GST

### Methods and Materials

Plasmid construction. A PCR product encoding the cytoplasmic domain of HCV core protein (amino acids 1-123) was cloned into pGEX2T (Amersham Pharmacia Biotech) to construct pGEXHCVc123, which encodes GST fused in frame N-terminally to HCV core protein residues 1-123. The plasmids expressing related GST fusion proteins to the HCV core protein fragments were constructed by using Quikchange mutagenesis (Stratagene) of pGEXHCVc123 or related plasmids and are described in Table 1. The PCR product encoding the helicase domain of human protein DDX3X (DBX), amino acids 168-582 of DDX3X, was cloned into pET23a to construct pET23aDBX168-582. The generated vector encodes the DDX3X helicase domain with an N-terminal His₆ tag. PCR products encoding N-terminal GST fusions in frame with HCV core protein residues 1-34 (GSTHCVc34) or 1-36 (GSTHCVc36) were cloned into the mammalian expression vector pCMVTag1 (Stratagene) to generate pCMVGSTHCVc34, pCMVGSTHCVc35 and pCMVGSTHCVc36. The plasmids encode fusion proteins that include an N-terminal FLAG epitope tag, GST, and HCV core protein fragments at the C terminus. The resulting mammalian expression vectors utilize the CMV transcription promoter. All plasmid sequences were confirmed by DNA sequencing.

In vitro interaction of HCV core protein fragments with DDX3X. The in vitro protein-protein interaction assays were carried out using GST pull-down assays (ProFound, Pierce). Briefly, E. coli BL21 cells carrying expression plasmids (Table 1) were treated with 0.3 mM isopropyl-β-D-thiogalactopyranoside (IPTG) to induce fusion protein expression. The bacterial cells were harvested, pelleted, and resuspended in 1 mL of phosphate buffered saline (PBS) per 5 mL of bacterial culture. The cells were then disrupted by sonication on ice. Triton X-100 was added to a final concentration of 0.01%, and the proteins purified using glutathione resin. Purified GST fusion proteins (1 μM in 1 mL) were mixed gently with 0.1 mL bed volume of reduced glutathione-Sepharose 4B beads (Pharmacia) for 60 min at 4° C. The beads were collected by brief centrifugation and washed three times with PBS containing 0.01% Triton X-100. Purified DDX3X helicase domain (3 μM) was added to the beads in a volume of 1 mL and incubated for 3 hrs at 20° C. under gentle rotation. The beads were washed 3 times with PBS containing 0.01% Triton X-100 per wash. Proteins bound to the beads were eluted in sodium dodecyl sulfate-polyacrylamide (SDS-PAGE) sample buffer, fractionated by SDS-PAGE (10% polyacrylamide gel) and stained by Coomassie blue.

In vitro binding analysis of GST-HCV core fusion proteins and endogenous DDX3X. HCV core protein fragments expressed as GST fusion proteins from the pGST expression vectors (Table 1) were purified as described above. For the in vitro binding assay, 0.1 ml of glutathione-Sepharose 4B beads (Pharmacia) containing various GST fusion proteins (10 μg) were incubated with HeLa cell extracts (500 μg) at 4° C. overnight under gentle rotation (You et al., 1999). The beads were washed four times with 1 mL of PBS containing 0.01 Triton X-100 per wash. Proteins bound to the beads were eluted by SDS-PAGE sample buffer, fractionated by SDS-PAGE (10% polyacrylamide gel) and processed for Western blot analysis. Detection of DDX3X was performed with rabbit anti-DBX antiserum (ProSci, Inc).

Cells and transfections. For HCV replication inhibition studies, the HCV genotype 1b strain N subgenomic replicon NNeo/C-5B was used in the Huh7 cell line (Masanori et al., 2002). The cell line was and was routinely grown in DMEM media supplemented with nonessential amino acids, penicillin, streptomycin, and 10% FBS (Omega Scientific) as described in (Masanori, 2002). If necessary, 500 μg/mL G418-active ingredient (geneticin, GIBCO_Invitrogen) was added into the medium. For inhibition assays, replicon cells were transfected with plasmids encoding N-terminal GST fusions to HCV core peptides using FuGENE 6 (Roche) using Roche-supplied protocols. In order to normalize the transfection efficiency, the plasmid pCMVLuciferase expressing firefly luciferase was cotransfected with the plasmids encoding the GST-HCV core peptide fusion proteins (pCMVGSTHCVc34 or pCMVGSTHCVc36). Briefly, about 3×10⁵ replicon cells/10 mL medium were plated into each 10 cm plate 1 day prior to transfection. For each transfection, up to 6 μg of DNA was mixed with 18 μL of FuGENE 6 reagent diluted in 600 μl of Optimem media (Gibco BRL) and incubated for 20 min at room temperature. The DNA-FuGENE solution was than added directly to the cells. After 48 hrs, the cells were collected and assayed for luciferase activity using standard methods and HCV replicon RNA levels.

Western analysis of fusion protein expression in transiently transfected Huh7 cells. Huh7 cells harboring the HCV replicon transiently transfected with plasmids pCMVGSTHCVc34, pCMVGSTHCVc35, or pCMVGSTHCVc36, as described above, were assayed for GST-HCV core peptide fusion protein expression by Western blot. Briefly, proteins were resolved by sodium dodecyl sulfate-polyacrylamide gel electrophoresis (SDS-PAGE) using 10% polyacrylamide gels and the Tris-glycine buffer system (25 mM Tris, 192 mM Glycine, 0.1% SDS). For Western blot analysis, the separated proteins were then electroblotted onto PVDF western blotting membrane (Roche). After blocking for 1 h with 5% nonfat milk, the membrane were probed with anti-GST primary antibody (1:500 final dilution) (Abeam), washed with TBST (125 mM NaCl; 25 mM Tris pH 8.0; 0.1% Tween-20) 3 times, 10 min/per wash), and then incubated with alkaline phosphatase conjugated anti-IgG (1:1000 final dilution). After the unbound antibody was removed by washing, the blots were developed utilizing the BCIP/NBT detection kit (Promega).

Northern analysis. Total cellular RNAs were extracted from Huh7 cells by using the TRIzol reagent (Gibco-BRL) and were quantified using a Nanodrop (Manufacturer) at 260 nm. The resulting total cellular RNA samples (10 μg) were then fractionated on a 1.0% TAE gel (Malek et al., 2005). The RNA was transferred from the gel to a nylon membrane by using 10×SSC (1.5 M NaCl, 0.15 M sodium citrate), and immobilized on the membrane by UV cross-linking (Stratagene). For monitoring HCV replicon expression, α-actin mRNA levels were used as an internal control in the Northern blot analyses as described by Jopling et al (Jopling et al., 2005). DNA probes specific to the HCV replicon and α-actin were [³²P] labeled on their 5′ ends using the RadPrime DNA labeling system (Invitrogen). Hybridization was carried out in Expresshyb solution (CloneTech) for 1 hour at 65° C. The membrane was washed 3×10 min at 55° C. with 0.1×SSC/0.1% SDS. Band intensities on the probed membranes were recorded by STORM phosphorimager analysis (Molecular Dynamics).

### Results

An HCV replicon assay in Huh7 cells was devised to monitor the levels of HCV replicon RNA that remains when the cells express protein fusions to fragments of the HCV core protein. When the Huh7 cells were transiently transfected with plasmids encoding a GST fusion to HCV core protein amino acids 1-36, the levels of the HCV replicon RNA dropped precipitously after one day (FIG. 2). Stable transfection of a plasmid encoding a GST fusion to HCV core protein amino acids 1-36 also resulted in very low levels of HCV replicon RNA after several days of cell culture. The specificity of inhibition of HCV replicon replication was assessed by measuring effects of fusions of shorter fragments of the HCV core protein to GST. No inhibition was observed when cells were transiently or stably transfected with plasmids that encoded a GST fusion to the fragment of the HCV core protein comprising amino acids 1-34 (FIG. 2) or amino acids 1-35.

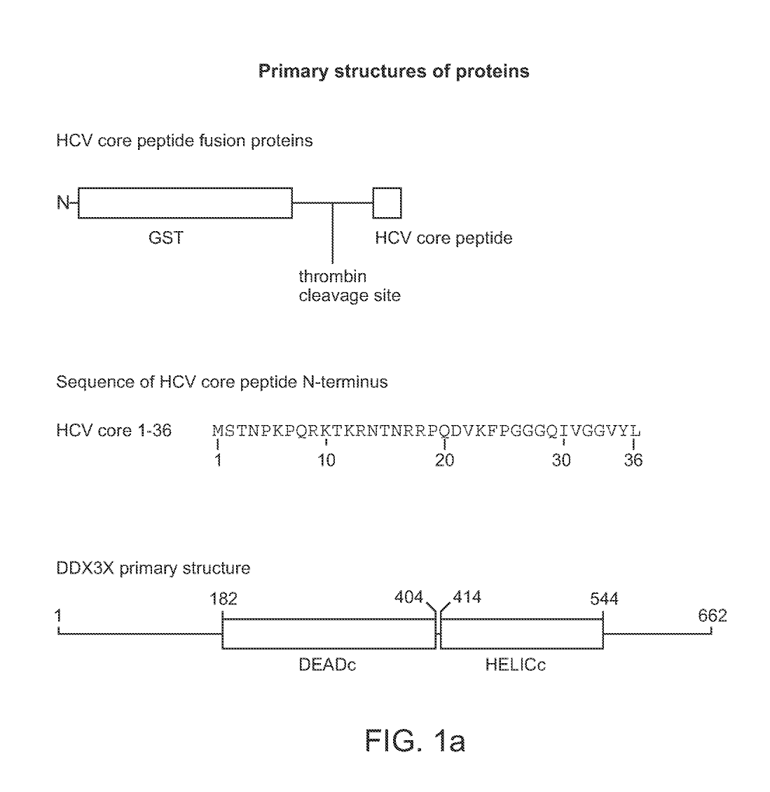

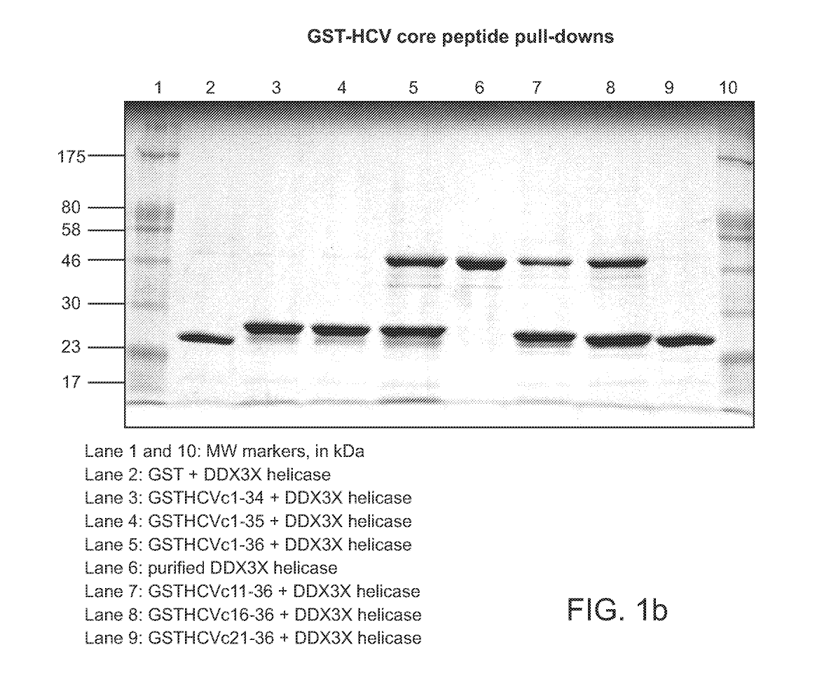

FIG. 1 depicts in vitro interactions between various N-terminal fragments of the HCV core protein and the helicase domain of human DDX3X. FIG. 1a: Schematic of primary structures of the different GST fusions to the N-terminal fragments of the HCV core protein. Also shown is the schematic primary structure of DDX3X, showing the DEADc and HELICc domains of the DEAD-box helicase. Based on DDX3X NCBI reference sequence NP_{—}001347.3. FIG. 1b: sodium dodecyl sulfate (SDS) denaturing gel of GST pull-down experiments with glutathione beads with various protein constructs shown in panel a.

FIG. 2 shows inhibition of HCV replicon replication in human Huh7 cells due to expression of an N-terminal fragment of the HCV core protein. FIG. 2a: Diagram of the HCV replicon NNeo/C-5B (Ikeda, 2002) used in the experiment. The plasmid encodes the 5′-UTR of genotype 1b HCV-N and the immediately downstream sequence encoding the N-terminal 12 amino acids of the core protein fused in-frame to the selectable marker, Neo. The Neo gene is followed by the IRES of EMCV fused to the full genotype 1b HCV-N polyprotein sequence and 3′-UTR (Ikeda et al., 2002). FIG. 2b: Northern blot analysis of HCV replicon NNeo/C-5B remaining in the Huh7 cells 1 day after transient transfection with the GST fusion protein encoding plasmids. Northern blots of actin mRNA levels serve as a loading control.

The following Genbank and NCBI accession numbers provide amino acid sequences for human protein DDX3X, and nucleotide sequences for nucleic acids encoding human DDX3X. 1) GenBank Accession Nos. NM_{—}001356 XM_{—}001126185 XM_{—}001129278; 5433 base pairs; mRNA; version NM_{—}001356.3; GI: 87196350; Homo sapiens DEAD box polypeptide 3; 2) GenBank Accession No. AB451220; AB451220.1; 1989 base pairs; mRNA; GI:197692140; Homo sapiens DDX3X mRNA for ATP-dependent RNA helicase DDX3X; 3) GenBank Accession No. AK304689; version AK304689.1; GI:194388151; 2349 base pairs; mRNA; Homo sapiens cDNA F1160399; 4) GenBank Accession No. AK304661; version AK304661.1; GI:194388103; 1592 base pairs; mRNA; Homo sapiens cDNA F1152848 complete cds; 5) GenBank Accession No. AK297159; version AK297159.1; GI:194386185; 2288 base pairs; mRNA; Homo sapiens cDNA F1160675 complete cds; 6) GenBank Accession No. AK296906; version AK296906.1; GI:194384603; 1051 base pairs; mRNA; Homo sapiens cDNA F1155031 complete cds; 7) GenBank Accession No. BC011819; version BC011819.2; GI:33873845; 2499 base pairs; mRNA Homo sapiens DEAD box polypeptide 3.

Table 1. Plasmid constructs used in the study. All proteins include HCV core protein fragments fused in frame C-terminally to GST. Single-letter amino acid codes are shown for mutations.

TABLE 1

| | HCV core protein fragment |
|---|---|
| Plasmid | (amino acids) |
| pGSTHCVc115 | 1-115 |
| pGSTHCVc115Y35R | 1-115, Y35R (Y35 mutated to R) |
| pGSTHCVc115Y35A/L36A/ | 1-115, Y35A, L36A, L37A, P38A |
| L37A/P38A | |
| pGSTHCVc115VY34/35AA | 1-115, V34A, Y35A |
| pCMVGSTHCVc40 | 1-40 |
| pCMVGSTHCVc36 | 1-36 |
| pCMVGSTHCVc35 | 1-35 |
| pCMVGSTHCVc34 | 1-34 |
| pGSTHCVc6-36 | 6-36 |
| pGSTHCVc11-36 | 11-36 |
| pGSTHCVc16-36 | 16-36 |
| pGSTHCVc21-36 | 21-36 |
| pGSTHCVc16-34 | 16-34 |
| pGSTHCVc16-35 | 16-35 |

### Example 2

### High-Throughput Screen for Small Molecule Compounds that Disrupt the Interaction Between DDX3X and HCV Core Protein N-Terminal Residues 16-36

Fusion proteins of GST to HCV core protein fragments encompassing amino acid residues 16-36 bind tightly to the helicase domain of DDX3X, as described above. In contrast, fusion proteins of GST to fragments of HCV core protein lacking amino acid 36, or with mutations at either residue 34 or 35 do not bind to the DDX3X helicase domain. The tight clustering of amino acids in the N-terminal fragment of the HCV core protein, in residues 34-36, indicate that there is a “hot spot” for the binding interface between DDX3X and the HCV core protein that contributes a significant proportion of the binding energy (see Wells and McClendon (2007) Nature 450, 1001-1009).

A fluorescence polarization assay (Roehrl, 2004) may be used in a high-throughput screen for small molecules that disrupt or prevent the interaction between DDX3X and the HCV core protein, where the fluorophore is attached to short peptides of the HCV core protein encompassing amino acids 16-36. Attachment of the fluorescent probe to the short peptide can be carried out using any number of methods that are well known to those skilled in the art. When the fluorophore-tagged HCV core protein peptide is bound to the much larger DDX3X helicase domain, excitation of the fluorophore with polarized light will result in polarized emission from the dye. If a small molecule prevents or disrupts binding of the fluorescent HCV core peptide to the DDX3X helicase domain, excitation of the fluorophore with polarized light will result in much less polarized emission from the dye, due to rapid dynamics of the unbound peptide. Deletion of amino acid 36 or mutation of amino acids 34 or 35 could serve as controls for the behavior of the fluorophore-tagged peptides in solution. Emission from these peptides should not show an increase in polarization in the presence of the DDX3X helicase domain.

### Example 3

### Design of Peptidomimetics that Disrupt the Interaction Between DDX3X and HCV Core Protein N-Terminal Residues 16-36

As noted in Example 2, three amino acids within the HCV core protein, residues 34-36, define a potential “hot spot” in the protein interaction surface with the DDX3X helicase domain. This small sequence can be used in the design of a cyclic peptide or peptidomimetic library to screen for inhibitors of the interaction between the HCV core protein and DDX3X. The synthesis of cyclic peptide libraries is a well-established methodology (de Vega, 2007). Cyclic peptides or peptidomimetics can be screened using the fluorescence polarization assay described in Example 2 above.

### REFERENCES

Ariumi Y, Kuroki M, Abe K, Dansako H, Ikeda M, Wakita T, Kato N. DDX3 DEAD-box RNA helicase is required for hepatitis C virus RNA replication. J. Virol. 2007 December; 81(24):13922-6. Epub 2007 Sep. 12.
Boni S, Layergne J P, Boulant S, Cahour A. (2005) Hepatitis C virus core protein acts as a trans-modulating factor on internal translation initiation of the viral RNA. J. Biol. Chem. 280, 17737-48.
Bourne N, Pyles R B, Yi M, Veselenak R L, Davis M M, Lemon S M (2005) Screening for hepatitis C virus antiviral activity with a cell-based secreted alkaline phosphatase reporter replicon system. Antiviral Res. 67, 76-82.
de Vega M J, Martín-Martínez M, González-Muñiz R (2007) Modulation of protein-protein interactions by stabilizing/mimicking protein secondary structure elements. Curr Top Med. Chem. 7, 33-62.
Fraser C S, Doudna J A (2007) Structural and mechanistic insights into hepatitis C viral translation initiation. Nat Rev Microbiol. 5, 29-38.
Gao L, Aizaki H, He J W, Lai M M (2004) Interactions between viral nonstructural proteins and host protein hVAP-33 mediate the formation of hepatitis C virus RNA replication complex on lipid raft. J. Virol. 78, 3480-8.
Graziani R, Paonessa G (2004) Dominant negative effect of wild-type NS5A on NS5A-adapted subgenomic hepatitis C virus RNA replicon. J Gen Virol. 85, 1867-75.
Ikeda M, Yi M K, Li K, and Lemon S M (2002) Selectable Subgenomic and Genome-Length Dicistronic RNAs Derived from an Infectious Molecular Clone of the HCV-N Strain of Hepatitis C Virus Replicate Efficiently in Cultured Huh7 Cells. J. Virol. 76, 2997-3006.
Ji H, Fraser C S, Yu Y, Leary J, Doudna J A. Coordinated assembly of human translation initiation complexes by the hepatitis C virus internal ribosome entry site RNA. Proc Natl Acad Sci USA. 2004 Dec. 7; 101(49):16990-5. Epub 2004 Nov. 24.
Jopling C L, Yi M, Lancaster A M, Lemon S M, Sarnow P. (2005) Modulation of hepatitis C virus RNA abundance by a liver-specific MicroRNA. Science 309, 1577-81.
Lai M C, Lee Y H, Tarn W Y. The DEAD-box RNA helicase DDX3 associates with export messenger ribonucleoproteins as well as tip-associated protein and participates in translational control. Mol Biol Cell. 2008 September; 19(9):3847-58. Epub 2008 Jul. 2.
Lee C S, Dias A P, Jedrychowski M, Patel A H, Hsu J L, Reed R. Human DDX3 functions in translation and interacts with the translation initiation factor eIF3. Nucleic Acids Res. 2008 August; 36(14):4708-18. Epub 2008 Jul. 15
Lukaysky P J. Structure and function of HCV IRES domains. Virus Res. 2008 Jul. 30. [Epub ahead of print]
Mamiya N, Worman H J. Hepatitis C virus core protein binds to a DEAD box RNA helicase. J Biol. Chem. 1999 May 28; 274(22):15751-6.
Masek T, Vopalensky V, Suchomelova P, Pospisek M. (2005) Denaturing RNA electrophoresis in TAE agarose gels. Anal Biochem. 336, 46-50.
McGivern, D R and Lemon, S M (2009) Tumor Suppressors, Chromosomal Instability, and Hepatitis C Virus-Associated Liver Cancer. Annu. Rev. Pathol. Mech. Dis. 4, 399-415.
Owsianka A M, Patel A H. Hepatitis C virus core protein interacts with a human DEAD box protein DDX3. Virology. 1999 May 10; 257(2):330-40.
Randall G, Panis M, Cooper J D, Tellinghuisen T L, Sukhodolets K E, Pfeffer S, Landthaler M, Landgraf P, Kan S, Lindenbach B D, Chien M, Weir D B, Russo J J, Ju J, Brownstein M J, Sheridan R, Sander C, Zavolan M, Tuschl T, Rice C M (2007) Cellular cofactors affecting hepatitis C virus infection and replication. Proc Natl Acad Sci USA. 104, 12884-9.
Roehrl, M. H. A., Wang, J. Y., and Wagner, G. (2004) A general framework for development and data analysis of competitive high-throughput screens for small-molecule inhibitors of protein-protein interactions by fluorescence polarization. Biochemistry 43, 16056-16066.
Shimoike T, Koyama C, Murakami K, Suzuki R, Matsuura Y, Miyamura T, Suzuki T. Down-regulation of the internal ribosome entry site (IRES)-mediated translation of the hepatitis C virus: critical role of binding of the stem-loop 111d domain of IRES and the viral core protein. Virology. 2006 Feb. 20; 345(2):434-45. Epub 2005 Nov. 17.
Stone M, Jia S, Heo W D, Meyer T, Konan K V (2007) Participation of rab5, an early endosome protein, in hepatitis C virus RNA replication machinery. J. Virol. 81, 4551-63.
Wang C, Gale M Jr, Keller B C, Huang H, Brown M S, Goldstein J L, Ye J (2005) Identification of FBL2 as a geranylgeranylated cellular protein required for hepatitis C virus RNA replication. Mol. Cell. 18, 425-34.
Yi M, Lemon S M (2004) Adaptive mutations producing efficient replication of genotype 1a hepatitis C virus RNA in normal Huh7 cells. J. Virol. 78, 7904-15.
You L R, Chen C M, Yeh T S, Tsai T Y, Mai R T, Lin C H, Lee Y H. Hepatitis C virus core protein interacts with cellular putative RNA helicase. J. Virol. 1999 April; 73(4):2841-53.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A nucleic acid comprising a nucleotide sequence encoding a DDX3X-binding HCV core fusion polypeptide, wherein the DDX3X-binding HCV core fusion polypeptide comprises a DDX3X-binding fragment of HCV core protein and a heterologous polypeptide.

2. The nucleic acid of claim 1, wherein the DDX3X-binding HCV core fragment comprises amino acids 34-36 of HCV core.

3. The nucleic acid of claim 1, wherein the DDX3X-binding HCV core fragment has a length of from about 5 amino acids to about 100 amino acids.

4. The nucleic acid of claim 1, wherein the heterologous polypeptide is glutathione-S-transferase, an Fc domain polypeptide, albumin, a halo-tag, or an antibody.

5. A recombinant expression vector comprising the nucleic acid of claim 1.

6. An isolated host cell genetically modified with the nucleic acid of claim 1, or the recombinant expression vector of claim 5.

7. A composition comprising:
a) the nucleic acid of claim 1; and
b) a pharmaceutically acceptable carrier.

8. A DDX3X-binding HCV core fusion polypeptide, wherein the DDX3X-binding HCV core fusion polypeptide comprises a DDX3X-binding fragment of HCV core protein and a heterologous polypeptide.

9. The DDX3X-binding HCV core fusion polypeptide of claim 8, wherein the DDX3X-binding HCV core fragment comprises amino acids 34-36 of HCV core.

10. The DDX3X-binding HCV core fusion polypeptide of claim 8, wherein the DDX3X-binding HCV core fragment has a length of from about 5 amino acids to about 100 amino acids.

11. The DDX3X-binding HCV core fusion polypeptide of claim 8, wherein the heterologous polypeptide is glutathione-S-transferase, an Fc domain polypeptide, glutathione-S-transferase, an Fc domain polypeptide, albumin, a halo-tag, or an antibody.

12. The DDX3X-binding HCV core fusion polypeptide of claim 8, wherein the DDX3X-binding HCV core fusion polypeptide is covalently modified with one or more of a non-proteinaceous water-soluble polymer, a lipophilic compound, and a carbohydrate.

13. A composition comprising:
a) the DDX3X-binding HCV core fusion polypeptide of claim 8; and
b) a pharmaceutically acceptable carrier.

14. A method of inhibiting replication of hepatitis C virus (HCV) in an HCV-infected cell, the method comprising contacting the cell with a nucleic acid comprising a nucleotide sequence encoding a DDX3X-binding HCV core fusion polypeptide, wherein the DDX3X-binding HCV core fusion polypeptide comprises a DDX3X-binding fragment of HCV core protein and a heterologous polypeptide.

15. The method of claim 15, wherein the DDX3X-binding HCV core fragment comprises amino acids 34-36 of HCV core.

16. The method of claim 15, wherein the DDX3X-binding HCV core fragment has a length of from about 5 amino acids to about 100 amino acids.

17. The method of claim 14, wherein the heterologous polypeptide is glutathione-S-transferase, an Fc domain polypeptide, glutathione-S-transferase, an Fc domain polypeptide, albumin, a halo-tag, or an antibody.

18. A method of treating a hepatitis C virus infection in an individual, the method comprising administering to the individual an effective amount of the nucleic acid of claim 1.

19. A method of treating a hepatitis C virus infection in an individual, the method comprising administering to the individual an effective amount of the DDX3X-binding HCV core fusion polypeptide of claim 8.

20. The method of claim 19, wherein the DDX3X-binding HCV core fusion polypeptide is administered in an amount effective to reduce HCV viral titers to fewer than about 5000 genome copies/mL serum.

21. The method of claim 19, wherein a sustained viral response is achieved.

22. The method of claim 19, wherein the method further comprises administering to the individual an effective amount of at least one additional therapeutic agent selected from an interferon, a cyclosporin, an interleukin, an HCV metalloprotease inhibitor, an HCV serine protease inhibitor, an HCV polymerase inhibitor, an HCV helicase inhibitor, an HCV NS4B protein inhibitor, an HCV entry inhibitor, an HCV assembly inhibitor, an HCV egress inhibitor, an HCV NS5A protein inhibitor, an HCV NS5B protein inhibitor, and an HCV replicon inhibitor.

23-32. (canceled)

33. An in vitro method for identifying a candidate agent for treating a hepatitis C virus (HCV) infection, the method comprising:
a) contacting a sample comprising a DDX3X polypeptide and a DDX3X-binding HCV core polypeptide with a test agent; and
b) determining the effect, if any, of the test agent on binding of the DDX3X polypeptide to the DDX3X-binding HCV core polypeptide, wherein a test agent that inhibits binding of the DDX3X polypeptide to the DDX3X-binding HCV core polypeptide by at least 10% is considered a candidate agent for treating an HCV infection.

34. (canceled)

35. The method of claim 33, wherein said sample is a cell-free sample.

36. The method of claim 33, wherein said DDX3X polypeptide and said DDX3X-binding HCV core polypeptide are present in a cell.

37-40. (canceled)

41. A stabilized DDX3X-binding Hepatitis C Virus (HCV) core polypeptide fragment, wherein the stabilized HCV core polypeptide fragment comprises amino acids 34-36 of HCV core, wherein the stabilized HCV core polypeptide fragment has a length of from about 5 amino acids to about 150 amino acids, and comprises one or more of a non-proteinaceous polymer, a lipophilic compound, or an oligosaccharide covalently linked to an amino acid of the HCV core polypeptide fragment.

42. The stabilized HCV core polypeptide fragment of claim 41, wherein the non-proteinaceous polymer is a poly(ethylene glycol) polymer, a polypropylene glycol, polylactic acid, polyglycolic acid, a polyoxyalkene, polyvinylalcohol, polyvinylpyrrolidone, cellulose, a cellulose derivative, dextran, or a dextran derivative.
